SEQUENCE LISTING (31 sequences)

SEQ ID NO: 1 - DNA, 33258 bp, Homo sapiens
  misc_feature 1..33258: note = chromosome 1, GRCh38.p12 Primary Assembly
  source 1..33258: mol_type = genomic DNA
ggggcctgggccggcgccgggtccggccgggcgctcagccagctgcgtaaactccgctggagcgcggcggcagagcaggtgagcgggcggtgccggggggtgcccaggccagggccctgtcgcctgcggcgctgagggcccggggtggggctgcgccctgagggccctgccctgccctccgcacgcctctggccacggtcccttccccggctgtgggtctgcggcccctgcgtgcgcagcgctcctggcctctgcggccagcgcgggggcggagagaggagagtgcccggcaggcggcggctgggccggcccggaactgggtcgtggaaggatcgcggggagcggccctcaggccttcggcctcactgcgtccccacttccctgcgcccgcctgccgccgagccccggctgggggtgggcgcggcgcgagcggttaaagggccggtgcatttaaaggagcggtgcacgtgggtctctgaggcgtgtagcaggcgggggcgttttgttcttcttctctctcgccggagacctccgttgcgccgagtccattcggcctctagcaccgggtcctgggcatgctttccccgggaaggaggcgcgcgggggctctgcccgcacgtgaggggcagggccgcaggctcaagcctagagccggtttctgttagcagcggtgtttggctgttttatcaggcatttccagcagtgaggagacagccagaagcaagcttttggagctgaaggaacctgagacagaagctagtcccccctctgaattttactgatgaagaaactgaggccacagagctaaagtgacttttcccaaggtcgcccaggtacgatatagcagagccaggcttcgaccccagtgtcctggcttctagatctgctgtccatccctccgagcagacctcacccctgtttattgccttaataagtattccctttgaaaggtatgaacggtgttgagtgaagtaactgcatccctatttacaaatggagaacctgagagcattccatagagacgattgtagactaacttaactcagaagcgacagcctggggttgccaaggctgtctacgaagtaacttgattaggaccgaccccagcttccagtaaggaagcctctgatgcctctgtagccaattctgcagacacctgagcctccaaggccttcagccaagacctttggcggtaattggagtctcgggataagctgcttcaggtgtgtgagcctcaggttcttctctcctgaatgtggttgtgggcagccggtgactggcgcaggtgcagaaggggcctggttcttggccccacctcagagctgcgtcctcacgacgcccacgttgagccttgggttccagggcagagactggagtgagggcttgggggcatgttgctttgaagtgggatggatgtatcaggtttttggggaaaactctgtaccctttggtgttgaagtgcccatgtgccaagtcttgagtccagcatgttcacatgtggggagtgagtggcttgttcctgtctatttgaaagagcagcaaggaggaggaggagcaagggctaggggctgctgctggggtgcctggagctgtggtgcataatgtcacacctgtctcccctccgtagctgctcaccgtccccccaaggggggtttgcctcttgcctactttggcctttctctgttatcgatgttaataatgacatatatctcgcttatgagttggtcataataaaaagctatcttgtacagaatattagaatttaagatcttaagaatttcaatgacactgaaatagtttattattacctttttacagaagaggaaacaagttcagggagttaagcagctagtccagttatgtggcttcagtgctttaagcaccaggatttgaacatagctggctacactgtctttatctcttgagtttttgcgcaggaggttcctgtattcaactcctacccgtgtctctccactactgctgggaaagttttgtggagtccccatgagcaacttcctgacaaacaaacaaaatttttttaaagaaaccaaagcagtgtgtgtaggtcacatgcagtgtgtctaatgaaaacatctctggcgggttttcagctgttgctttgactttcggacactgtttagttggggactgataagacagcaaatatttctgcaagtattcccacctgttctattcccagctgccacagctgcggaaaggcgggggtgaggctgagaggccccgagaggaacattttccactgggctccaatcctggagatgggatgaccatcatgttaatgtctggagaaaagaatgatttcaggctgggtgctgtggctcatgtctgtaatcccagcactttgggaggccgaggtgggtggatcacctgaggtcggaagtttgagaccagcctgaccaacatggagaaaccccatctttactaaaaatacaaaattagccgggcgtggtggcacatgcctgtaatcccagctactcaggaggctgaggcaggagaatcgcttgaacccaggaggcggaggttgcagtgagccgagatcgggccatggcactccagcctgggcaacaagagcaaaactccatctcaaaaaaaaaaaaatggtttcacatcagtcctcaggaaagatcagatgtcagtgagggagatcatttcttgagagcctcttcactgagtgggagaatgggctgcttgttcatctttgtgaaaattctagaacgggaagaacaattcaaagggtgtccaccattctgctgtaccttaaccagaaacttactggactctttttaaaataaaagtaattcatgtttattctagaaaattagggaaaaaaaattttttttgagatggagtttcactcttgttgcccaggctggagtgcgatggtatgatctcagctaactgcaacctctgcttgccgggttctagcgactctcctgcctcagcctcctgggtagctgggattacaggtgcctgccaccactcccagctaatttttgtatttttattagaggcggggtttcaccatgttggccaggctggtctcaaactcctgacctcaggtgatccgccaaccttggcctcccaaaatgctggtattacaagcataagccattgcgctgggctgagataaccactcttaacatgcatttccttccacactgttcacatatgcatatttatctattaaaacaatagagggagggaaccgtaggtgaagtttgtgtatcctgttttttctgttatgccttcagaattttccttttattgagtactcatggaaaagcagatttgatggctgtgtagagcatttgaattatttatcctaccagtcccacagcaggacactttcccaccctcccttttgccccagagaagcagtgccctctgtcctccccatgcccatatgtgggcactccccaccatggagccaaacctacctgggcaagtagcagagggagagcagagtgagccctgggggcaggagagagacttgagagttttgaggtgacagatgagctggtgagtgagtgattagggagcatttcttgacacatacctgcccgtggtgaaggcatgtgtcttgtgagtgtgctcccagaaagcctgtgtagtgtgtggtgggcctgcctgtgtgaccaaaccctggccactgggtacgtgaccctcacaagtgctgactgggctgagaagagctccttgatgggcagtttggagacttgagttgtaactgtggcttttggccatgggacattaactgattacttttgcccctctaggcttcagttgtccttaattataatacagggagctgactaggtggcgttgatggcctttacggtccttgccagctctgacattgtcctatggatatgtcctttcatttgataatatgtttacgtggccatagtgcctggggctgggccgggaatggaaacttgatctctggggcctggcctttgaagccagttcatgtgtctggtggttcagcagatccgtaactttccaagaggcacatccataggctaccgtgtcctttctcactgtgtccctcctccatttcatcttctttataactacgacttattgaacatctactgtgtgctggacactttacaggttatctctaggttttacgataatcttgcaaggtatgcctgttctgctttttacagcagaggaaatgagctgtgtcagattagactgtctgaggcctcttggccagggagtatgtggttcaaatcacataggcaggcgatctgaaccctgtcagtctccaaagcctctgcttttgaccgctgacttgctgctgcttgtttaaaaataaatgtgtttctggagcctactccagaggggcgtgctaggggctccctctcccacttccccacaaaccacccttttccctggctgcttcaggaaatgagagaactctgcctgggccccaggcacttctgagtgggacagggctgttagaggtaagtctagagcctggcccaaaattcaggaggccccatcagagggcccctggggcctgtggtccgggagggtggtagggcagtacctcacttccctttgagactcaggccccagctctggcttaggccagggagaaccatccccaagtggtatgtgttactatatgagctgagatggatggtcagctggaccaaatacatagtcgggtacccagggccagggggaggaaggtgagcagggaagctgtgggcaattgtctgggtatcacctgaccttagcaaactcttccttgttttaagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtaagtgcagtgactcccaacctgcttttgaaccctctttttccattaggattttctccgtggaggcagatttccatgggagtttgctgtggcattttgaaatctgtttcttacctagttccattggccttaaatgttaaggccaaagcctttacatttctctgtaatgaaaagaaggtcgaggaaattgggtcattgggtttccataatgattgcaggaactgctgacacaagcacggctggggagattctctaggtcagactcccttggtttggctaattcagcagtttgatcccattcagctgattaatgggaatgtgcagtggcttctttggatgtttgattttgcatcctaatccaaagcagctatcagcctcagcacttccttgttggaaggctttccagaacgtagtctatgttggacacttccttctgcctctctgcattttcctgccacttctctagagaatggggtgcagggggtgggagacggggaaagctggtcgctgagtggctgatgggacttgacatcacccagccccacccccacctgcccgtgagtcagcctccggggagagttcatcgcgtcaccggcactctaatgtggacagacacctagcagtgttgtttatctgcacacgtttgggtggtgatttttccctccaaggatttcagagcaccagcaggcttcagagcagacttaggtggcttgcaaagcaggccctcaggaattcagagggtagcagaagtccatcccagatgctctgttttccttcaggagctaggtaaatcagaggggctgagggacaaatgaaaaaagttacagcctttgagtcccatctgctcctcctggccaatgagaggggatctgggaggggcagatgtagaggaaaatctgtctaaatgttgatgctcgttattttcctttaaagaattaatagcctaaaataaaccctacagatacagtctgtgtttattatggcgacttagagaaatgcagaaaaatatcaagaaaataaaaaccactcttggttctaccatgcaaagataatcatctttaatgttttgtaatatttccgatcttttatatacatacattttataaggacattcagatgatcaggttcgtaaagttttatgttcggttaaatttaacagcgtgtcattgttcaggttattaaatgtttgaaataagatttttggtggtcctgtcacagtctccatgaagtagcatttcaggatcgaaaggtatgctgtgtttaaagtgttgattcttactcctttcagttaaggccagtgcagtttgtccaggtagtgactgagacccagtttttccacactctcctccgcagtgggcattgttttgggcctttttcagcccaagagctctcttctccccatgccgctctgctggtctgagatttttccactcctcctcctccctagttgctctctgaccagactctaggtattcaggagaaagtgttcattgtctcactctctcatgtggcaatcaagtagtgccaagcagtgagagggtgaaggtgggtgggtgagggacactcaccttgctgagaaagggccccagcctgttcgggtgattataaagcagagacagtgccaggaaaagtctgacactggctgagaatcacccggggaccaaccatcccgaatgcggatccctgacactgggtgaggatggagcttggagatctgcattgttaataagcagcctagcagagtggtgaagagtccagacacactacctaggtccaagggtaaccttgagctaattactttttgagcctctgtttcctcatcagtaccatggggaagaatagtagcaccttgctccaggatgtttagtgccggctaagggctcagcaggtgctggtccatctccaccagcccccagtggcctgggccacctttgagaaacagtgatcctaagggattcagcatttcctaagttggtgcctcccacctgtcacccccaccccaccaggctaggagggttgtgattagagggtgcccttgctgtgacagctgagactagctcttccctgattattccttaatgacagctctctccttccctgctttcttgaagtcttggtcctcgttgttgtgggcacagcttcaggggaggccttggaggaatttttgaaagtggaatgagggaagcagcctgctcaagggaacacttgttttctggtgaggaggccgcatgtatgaatgacgtttgtgggttagaaagcatgttttgtagtttttccttgtttcttcctgaagacatgtcaggtcttgatgagaccgggcctgggcacagggcaggcagtcagcgagtgtggatgatgacgacagtggtcaccaggtcactgtctagaccaggtcactgtctagcgcagtgtcacatggaaagggtatggtcctttaaccctaccctccccagcacaactatcacagatgtcagggaacctctgctcacagaactgctttccagggattgtcttttttttctttttctttttctttcttttttttttgagacagagtctcactctgtcgcccaggctgaagtgcagtggtgcgatctcagctcactgcagcctccgcctcctgggttcaagtgattatcttgctacagccttctgagtagctgggattacaggtgcctgccaccatgtccagctaatttttgtatttttattagagacggggtttccccatgttggctaggctggtcttgaactcctgacctcaggtgatctgcccacctcagacaggcatgagcaccgcacccagccccagggagcgtcttattagtggttggcaactgaatggagacgtgggaattgtaaggaactgattctacttgatcctgggtcccctgcttctccatcttcacccacccatcagctccctttctcctttaaacaggcacctttgctctctgcttatccatttttgttgtgcattgctatttgggagcctaagaaacacaacatcctctgaatgctccagctgttgtgggtctgaagggtgagcctgccctctgtcattggaggctgcagcctgtggctttttaggtacagggactcccagaactgctcctccagtcatagcagagataaatcacaggagcttaagaggcatgggaagaacagagggaggagatcgtagcttccctgttcattcacacccaaaacaaaactgtcatactagaaaaggaggtattaaaagagccacctgtacagcctcgtatctcatccagcacactgctgcagatggaatattatgatttagcttgagaaaatgcagcaactctttgttgtggtgcccctctttgagtaagagtgaattccccattgccagagtggatagtgagggaaaccctgggtccaggcaggagtctgtttaggatttatctagtgaggctgagccagaggaggaccttacagttttttctcttcaatttcttttatttatttatttatttttgtagagatggggttttgccatgttacccaggctggtcttgaactcctgggctcaagcgatctgtctgcctcagcctcccaaactgttgggattacaggcgtgagccgagccaccatacccggcccttctcctgcatttccacctgataatttctctcatttccatagatgatgaaggaactaaagccaagaactttccaaggtcctgcagctctttgggggatgtgaagctgtgctctatttgtatggattttgctggttcccagaacttccctgtggccctggggcctagtctgagggtactctgagtgaagagggaggagggcccacacctcttctgcaaaggctgcttttgtaaagttcacttcagttcacatcttcctcctggtcagaaagcttcgggggctctcctctgctgcattaagctcttactcctccatcaggcaccaaactcctccctggcatggcccatcctaccaggtccccacacttgagccacatccaattgctcgatattatcaggataggttatgttatgttcccaactcatatgtttacttaagtggttacctctttccagaatgagccccctcctccaaactctgcctggtgaaatattcctaacctttgcagcttcacatccctcttacttcttgtgacctgaggcatctactcctgacaactgatagactgtgtcccctcctgtcgggtgcattgtccttgtcactaccctcctggcttttagctggctttgcttcccgctgttgttactcctgtacttgtctcatctatcctaaacagaaggtgctgcaggctggggagtttgttcatgttgaaatccctgtgatggaggtgagcagaggcagtctctgcctgtgcctcttatttggggatgaagttaaagtccctgtaggaataatccaggccatagccggggttgctgtcttcagaaagaagggcagccacaggtcttgttaaggggattgaaattggctgacttggtggaaggaacctgcctgctttgtttaaaaaccacatatagctgagtgtagtggttcacactctgtaataccagtgctttgggaggctgaggcaggaggatcacttgaggccaggagtttgagactagcttgggcaacaacgtgagaccctcatttctacaaaatattttaaaaattagcctagtatggtggcgtgcatctgcagccctagctactgaggaggctgaagtgggagaattgcttgaacccaggagttcaaggctgcagtgagctatgattgcaccactgtactccaacgtaagtgaccagtgagaccctgtctctagaaataaaaataaaaaaaatcacatatattgtggggtgacttacttggagacgaactttcagcagagcgcacacctgctatccctgcccagggtgtgaagctcagccctgagggtctctggacagcgatcactcagcctctggacagcgatcactcagcctctggacagacagcgatcactcagcctctggacagcgatcactcagcctctggacagcgatcactcagcctctggacagcgataactcagcctctgtccccgtctgagatgttggcagggactgtcagatttgccaggcattgtttgaagttcttcccagcccagaaacctgcatgtgtagattttggtacactgggtcccccacttggtactactgtgtgaaaccccacttggcactgttttagggggcaggcttccctcctgtccccttggccttggccttcccctgggtcccgccctcagtggcacttccccacctcacacgtctgctctcatggcttaggtctccacttctaacctcaggagacctggtcctcagacacctcccagacagcttccccattttatcccatagacactcaaagggtgaaattcatggtctttcccgagactctcttctccggtcttccctgtcttagtccccacctggctgccattctagactgtgttttctctcctgcgtcaggtcctgcccttgacctcttgaccccttctggactctgccctcacttgcatccatcctgctgctgtcctcagctctcctcacctgccacagttgtctctgggggtcactttccctctctggtcagtggccagactgacttttataaacctggttcagatcttgtctgtcaagattgtcctcagggtgatgtgtgtctccttaacatggtgcctgagaccctgatcatctccactgcccgccccacagtgtgaggccctcactggaacattgtgccttctgcccttccctcctcctgggaaaaccagtctccatcagataggctcttctctagaaaacattcgtgatctctgagatttggttccacttttgtgcttctgcacctaccatcaaacacccggattgtatcatttgtcacattagatgatttttgttttgttttaagacaagggtgtttcttactcatctttttatccccagagcccagcatgatctttggtgcataatagatgcaccacagatgtttgctgattgaatgaatgagcacactgacagtttggagctgccctgactttcgtggctatgcgttttgccccctgggatgtgagtcacctcaggccagccccaggcaaggccgctgctgcctccatggtaactctcaaggcctcttgttttatggcagtcgtttgattgacaggcatctcttggaagcttttggggcaggacttgtgtccaagtctccaggtcgcctccagccaccccctgagtcctccactgcctttgtctcacaggaaagtggaacaaggtccttgtgctcctttttccaggtgcttccagaggcagggctatgctcacattcatggcctctgacagcgaggaagaagtgtgtgatgagcggacgtccctaatgtcggctgagagccccacgccgcgctcctgccaggagggcaggcagggcccagaggatggagagaacactgcccagtgggtaggtcccaccagcagctgggggccttcaaacaggtccctgcggctactgtaccttacagatgaaaaccagacattcattccctgatgcgggagggagaagggaagtaatgatgaggattggccgaaaaggtgggtggctggccatgatggaccttccatctgcagggtttcataggactgcgcattcacagccagagatggacttggcagtgggctgaaggacgctgtccactctgccaccttgggtttacctctctcatgcaggtcactgtttccactgtaataggagagtttgtttggatgcctgggtgctaggacaggtaacacagaagcttaggatggtagcaggggaagcattttttggcagatggccagacatggtaagtgtgagaggagtctgcctgatacacgattgacttttgagctggggatatttgggcttcactgtgatcattcagcccccaggggaggagattgtaacgttagaaagagtaggatatcgttgggagagccacttagttgtgtcctttctctcccgatcagggcagaacatctgaatttgcctgaaccctgttctctgttttgcccattatagaattaaaaaatgtctctgtgtggactgttttcttgcagccagtcttaatcctgcttgctgaaatttgagctcacttctccatgttctccttgagaacggaaccatcgtccctaagccctgagtgaaatcacaccagcttaaggccactgctctgccactcctcagccttttcttgtttgttatctccgggaagttttgtacactttggttgtttcagtttctgttcatgagtagtcttctttcttggctgaacgtctagattgggactctctctgcagagaaccggtactgaagcaactgtcattttcagtttttgtttcatttggctttttctttagctgttcacctcattagcaaggcagcccatgaccttgacttgccacagttccaaaacacaaattcttacagatcggtttgtgctagtgtctggcaggtgtcctgccctccctcgttacctcctcatttgtgcctgcccaccttcccagagcctgcgtcttctcagatgcttaacacctgtttagcctctctagttcagagctacaaatttacatgcttgattctgtggggcagaaagttcaaagtaatttcttcctctgcaaattcccagtatcttagtcacacgcaaagagagtgtccctgtgcactgactcctctagctagtgatttgtcagccaaaaatgtttatttatctcctggcctgtttcctcccatatcagtatggccacatgaacagaattgagtgacctcctgagtccctgtattaggaaggggaaagatcttttgattcattaaccattaagttgattcattaaccattaagtcttgggcctgcagaccatagcaaccttccttccttcatttatggtgcttcatccagctccaaatcttctctactttgtcctcacaaacttttcatatgccctagtagctcatagactgctccttatatctggaaagcaacattcaaacttctcatttctggttccaaaaatccgtgcattacatggataggctgccgtgggggacattctgcggccctcacgatgtggtttcccacagagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctgtgagttggggggctggggggagcagggtggggtgagggctgagttgccagggggtggggggcgcagcagcctgtgttggtcactgtacctgcagctccacaccagcagcggtaaagagcagggatgaagaaccgcccaggttcatggcctggctcactgcctcctggattgtgacctacttgggcatgcttttaacatccctatgcctcagcttccttgttcgtataatgggttgataacgcagttactgggagaattaagtgagttaatatgagtgaagggcttagaagagtgtctactgcacgtgagtgctcaggcaagctggatcctgctgcagaaagcaagctcttgatcctgggcatggctgtgccactgatccctgtgtgactgcaaacaaatcacttcctctctgagtctctgcttccctgaatgtgaaacaaggtggttggaccagatatttctcagctcacttccagccttgtgaggaagacttataaagcctttcgtttattttagtaaaatacatgcagaggcagcagcgtagaaaaatgagaagcttcctccacttcttccccctcccctttctgtggtcctcactgctaagcaccttctgtaaacttttttttttttttttaaagttagggatttttgtttcatttcgtgtgtgttggttttttttgttgttgttgtttcttttaaagaaaggaataaggccaggtgtggtgtctcatgcctgtaatcccagcactttgggagactgaggtgagaggattatttgagcccaggagtttgagaccagcctgggaaatgtggcgagaccctgtctgtacaaaaaatgcaaaaattagccaggtgtggtggtacatgcctgtagtctcagctacttgggagactgaggtggaagaacacctgagcccagaagtcgaggctgcagtgagccatgattgcgccactgcactgcagcctcagcaacagagtgagaccctgtctcaaaatttttttaaaaaattaaaaaagaagtagagtcccatcctcagaaagcttatagtgtgtgggggattcagcgcagaacaggtgaaagcatggagagaatgcagccagcggtttgtttgcagcagtccaggctgggaagagtgaggtttgagtgaattgcttcctgtgtctgcttcctgagcttatgagctgcaaggacagcagttgcttcagcggatgggggtcgggtagtagcaggtggaggagtgctgggctgggtggagctggtggagaggtgtgggtgggtgggggaatgagaactggatgggtgagagaagtgcctagggagcctttaatccctgtgggggtggggaaagcagcagggaggtcatctagccctcgtcctcactgctgcactgggcccagttggcaggctgagagccacaggtctgtggtcagggtgccaggaaatgagctggaggacaggaactgctcatggggatggtgcccgcactccatcagggcagcatgtgggcagcatgggcatcccaggcacctcccctagcaggtccagaatcactcaaggtggggagcctcgaggagcagtcagggccgggagcatcagccctttgccttctccctcagcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaaggtgaggccctggccctgccctccagccacgcttctctccgtctgccccacaccatggcggcagggcccgtgaaacagccgcctttagaaaaacacaaattagaggaaaatagacccagattttttgtactcctccccaccccatcctgtctcccaccgtggatgacctaatactgttgtcttttatttttatttattttctttttcttgaaacatggtctcactccattgcccaggctggagtgcagtggtgcgatcatgactcactgcagcctcaacctcctgggctcaagttctcccacccagcccctcaagtagctaggactacaggtttgcaccaccatacctggctaattaaaaaatttttttttgtgcaggctagatctcacagtgttgcccaggctggtctcaaactcctggactcaagtgatctcccaccttggcctcccaaagttctgggattacatgtgtgagccattgcatccagcctgttgtcttttaaatttacacattatcccacttgagttcctcattgcagtgttccaagcatcatttctcatatttcaaagttaattttgttttgcttctctttctgaagttctattttaggctcccctcaccccgatacttcccctgaagatttatttttagttttccttttccttttcgggcaaggatgtgcagaggccatgctgaggtcttgcagccctgggagacttttgggttgtagctgcctatagctgccgagtagccccagggagtagtggaagggcagatcccatctggccagaatcatgggcactgcctgtccccaaagatgccataagcttttagacagcggcttcaggcttttctcccaggtaaggggttgaacccctaacgatggaaaggaaattaagctgggcattacctattttaaaactgtttacacacaggtgcctcacagcattttttgttcaggccgctgccatccatggagcaggtagatagaagtgcagagtgcccaggctagagggatgggacagggacagtgcagggagggagctgagcccccttccagcgggggcagcagaggggaaagccatgggaggggctgcaggatgtgtcctgagctgaagcttatcaacaagtaatgagtaccagctgggcattgtggtgcacgcctgtggtcccaactacttgggagactgaggcaggaggatcgcctgaccccaggagttcaagtctagcctgggcaatgtaagaccctgtctctaaaaaaataataataaaataagtaacaattacctgtgtaactgtgacgaggcagggtttgaacattgccgctgggaggttggcagatggtgggaagcagggtggagggctgctggtttggagcagaggatacagattgcatggggtcaagctagaaattgcgtggcagatgtgaagagctggccccactgcgggcagtaggtgtctggtggccagtcccagaggctgtgaagaggggctcagccatctgtctagtagggcttccttggaggttccacgatacaggcagatggtggtggcccgggcagccaggtggtggctgggatgaagagggttggcaggtcccagaggcagccccttccccttttggctgtgtgtgcagcagggccgtggaggctgcttttagtccaggtagaccagggccacgctgaggtcccagtgggctgagctggtgactgatgagttggtcctcaggggtgaggctggtgggaagtgatgtcactgtcccgccgatggccagctaagggactgggttaggatcagccccctcttgtccttcactctcccatccttggccaggagaagaggaacaggtctttctgaggacctgcttgtagacctttgggtaggaggggacttcccaggttctctgttgaggccactctatctaaaatagcaccccagtgagtctcctatcactgtatcctaacattattttctccatggccctcatcattacctgctgatatactgtatgtttgtctatatgtcatctaacacccctcacactggaacacaatgcccgtgggcagagactttgctagccttggttccagagcctagaacagtgcctggcaagtaggagacacccagcattacctttctaagtgaaccagtagagatggggggagaccgcaaggctatgccggcagacctgagggagtcctgtctgcatgcgctgcaggatgacctgaggggaactccttggacttctgtgccctctttatctgtaaggtggccacctgatcccttccagcgtaggcatgaagtagcctaatgaagagcattcaggcttgggtatcagtctcaggatcctgggggccttagaatttgtggcgcttggggacaccttgtgatcgtgcaatttctgttgtctagttcatccatggctggttgatcatgtcttcactgatgctgctgttcctcttcacctatatctaccttgggtaagtgacagataagcagcagggtccctgggagcccctctccatgtggcacaagtggacatgggcatgaggacctgggcggggaaagatgaccatcgagctccagtcttccccagtgccagccgttttgggaacccaggcctccgtcgccctctctcatggccttgacacaggggagtggaagtggggctgcatggtggaccacatgtttctgtctcgttcctgatttaaaatgaacccttcatggagaaggctctctgtgaaccccagggggatagaaaccccccaaaatttacattctgatttttaggctaggcctgggtactttctggtttgtgggaaaaattatctgttctatcgccccttgatttgggatatcagcctgacccaggggcccaaagagactgggaggacaagagaaaacactttcccaaggacctttccatgtgcacagggtcttccaggtcatgcccatgcacatttctgtgatctgttccaagcatccccaccttgttttagaaaatgctgcaaatggtaaattgtaaggacagtgaaggtcggggaaggaaatgttagtaaagagggccaggttgggactgaatggtggtaaactgctaggctgtaatgcctccactgagtcccagtcacaggctccaccttggtcctgcagggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatggtaggtgggcagcaaggctggtgggggcagtgggggcgatgtccagggccaaatcgtccccagtgctgcacaaggagggcaggtgctgaagggcttgcatccctttctgcagaggcctgggtgggatccctcctgagagagtcgcctttgtaaaacagaggggggtccactatttctggaacactcctggtggtctagataaaacgcagtagtcactgagctcctcatttactttttttttttttgagatggagtcttgctctgtcgcccaggctggagtgtagtggcgccatcttggctgactgcaacctccgcctcccgggttcaagtgattctcctgcctcagcctcctgagtagataggattataggcatgtgccaccacgctgggctaatttttgtatttttagtagagatggggtttcaccatgttggccaggctgatctcgaactcctgaccttgtgatcggcccgcctcagcctcccaaagtactgggattacaggcatgagccactacacccagcctcattttccattattactgctatgctgattgagcaagtgcactgttaagcactggacacgctgtaagtgatttgttcatcaagacagtcctttgggtaccatgcatatacataaccccaaatgttagctgctatttgatattagcatgattatcattgccagtattgttacttccattttaaggttaaagaattggaggctcagagaagtgggactccccagcctggccaccgcgtctcgggtgcacagctcctccatgcttgcagttgcctgcgaggccctactctggctcacaccagggcctgctctaagttgtgactggagaatgagaatttgggatgccagcccagaggcaaggcatgctctgagagctccacccggggctcctgtgctacagggcaggctcttcttcagggggctgcccggggatagtttgacaaggatgtctctgtcttcctagatctcgtggctgtgctgtgtcccaaagggcctctgagaatgctggtagaaactgcccaggagagaaatgagcccatattccctgccctgatatactcatgtgagtgagccccccgtgcctctgcctgactcggggtcagcaggcagcctgtggggggacaggggcctgcttcctggccgtggctttcagagttgactgggcgatcccaggagggtctccactttcagaagccagggagggcagtatcttgttattacacagtaagaagcttagaaagttaggacaggaagcaggcatctgctgggatgtgctgcagtccctgacttcatcccgtccatcctccagcggcatgctgcggtgcaggttgcattcctgtgatcccgcagccacccctcagctctccaggctcttgagaagggactttggagagggattcttcagggcagggggtcggggagcaaggagcttctgggcttccttgacagcagcgtggctgattggcattaatcctaactgaagggaaggcacacgggatggcccctggcctcggggtcaatgtgtagagatttggacttacacatgcagtcaacaaaggcacatcaagtccccattttgtgacaggcactgtgctaggcattgggggacccagcaggaaagaagaccacagggtcccaggcctcatggagctcacggccctgtgattgtgatgccctcggtctgttgatggcggggcttaaatagcctgaatttctggagctctggcgtctgcaaggtggcctgggaaagagtttatggaacagctacagagttctaggtaccttcatgcagttgaggattcgagcccgtagaggagaatcgcctgcagcgtggccccacgggaaagcacattccaggcgcattccgaggatgagcggagaccatgtatggaaaggtagtgccaggactgtcatgagtgtcccagggctcgggggattcacccgtgaactgtgaggtcttggctctgatagacctggttcttatgctttaggaggggagacaaacagtaacagaatagacaaatgcaagagagagtgactctggacccctcccacaacggcctcctaacaatggagcatgagcagatacctgcaggatggagggtcctgtgcaggctttctgggacgcagactggccacctcccccaggccctgcaggcagccactgttagcaccgcctgagatgtgaaccttttctcctcccccagctgccatggtgtggacggttggcatggcgaagctggacccctcctctcagggtgccctccagctcccctacgacccggagatgggtgagtatcttggggagctaacagcctctcatcactggggggcagctccctacctgcacccagctctgctcggcctggcttccctgagaggcatgagttcaggaggggcagagggaaaggtccgttgaaaaccagccggacacatgcggcttgaagattcagcaagtgttggaccctcggtcctctgccagcctctgttgcatcgttctgctgggcgtgggtgggtggagtgggggaagccctggtgtcaggtgctggtgctcagggggaccccttcttggagctttgttccctggtaacactctgaccagctgttgtttctctctcttgttgtcccctcctcacggtgatgacggacatcttctcttcctggacacccagaagaagactcctatgacagttttggggagccttcataccccgaagtctttgagcctcccttgactggctacccaggggaggagctggaggaagaggaggaaagtaaggtgcccatgttcacacggcctgcttcagcctacggcgggagcggagacagagggtggaggctccctgcagcctgggtggaggagggcatgaggggaggggccccttttcccatcagaggcatctctgtgaaagtagaagatgcctgcagcgctggggtcttctcagcaggccccatgtagttgtccggcatgtattgagtatgggccacgtgcccgtgctgtgctgggtgaggcccagccctggtgggacccacaggctaaggagacacgggcagtaatcacatagactgagaagccaaggactatgaagggggccatggggttggggaggggcggcaggagagcatgccacggggcttcttgacctggttggcaggggtgagagaaagtcagctgaggaagtaactgctgagctgagctctgaaggttgagtcacagcagtcactagaggagaggagcacagggtggggagcatttcctgacagacagactcaggaatcagaggaagccggggcgggatgcagagagcagaagtgtgggagagccttgcaaacaggcctggagacatgcgaagataggagttcatcctggcgtcagtacacggtgcctgcctaacacccaatgccagcccactgctgcgtgccaggcagcaccctggagcagggagatgctgcactgtcgtaacagcccctgccttgagaggtgccttacgggagcagcctggtgacagtggcttggcatacaggactccagtgacacgggaggggcaagctagggaaagatcactctgcggtgggtctggaaggaggagcaggtgcgcaccctccaggcaggcttgggggaggtatttattccaaggccaactggtgtgctgcagaccaggagttagcacagatcccacggggcccgcaggactggcctccctccagacaccagccacaagctctagagggtctagatgccacttgtgcttctgaccggctgcaaatttagggctcccatgacccccttaggttcaataacttgctagaatgactcacagaactcaggaaagcactacacttaaaattgcagtttgttttttgttgtcgttttgttttggagacagggtctcgctctgttgcccaggctggagtgcagtagcacgattgtggctcactgcaaccttgacttcctgggctcaagtgatcctcccaccttggcctcctgagtagctgggattacaggcacgtgctaccacacctggctcatttatatttttagtagagacaaggttttgacttgttgcccgggctggtttcgaactcctgggctcaagtgatccacctgccttggccttccaaagtactgggattataggtctgagccacagcacccggccaaaattagttttattataagagatgcaactcaggaccagccaaatgaagagacagtgaagaagtaatgctgatggatcacacctggtgggggaaggaggacagctggggccaggagcaggagggacacctgcagggctggaagggcaggggaggtgggcctccatggtttgtgtttattgcataaccatttttattgtctacagtgagcaaagttatcctataaacaagtgtcagggaccattgcactaaagaaaacaaacgagagcattttggaagctctaatttcctgatcagtaatgggtagactaattcccagttatatttacctgttgtaaggtgaaaggttcttcagaggacctctgtcttggtgttatatgggcttttgaatgtactgaaattaaattccctaaaaatctgtgattcagacttcatactaaattgtacagcagtgcccagcccaaggccttgcatttctatttgttgttttctttactctctaagtgcccaacactggttttacctgagtttcagaactgcccgcttttctctgcccaggttgtaagtcacccagtccacaggtgtcccctgctttcccactggccactgatttggggaggcagctgtccatgtccccagtccacatcttagcttctagaggccaggtggggtgggctgggctgggcaagagcagctgggccttctgggccagagtttctcttctttttccattctgtgcacgcctcttcagtacgggttactgtctctcctcacacagggggcgtgaagcttggcctcggggacttcatcttctacagtgtgctggtgggcaaggcggctgccacgggcagcggggactggaataccacgctggcctgcttcgtggccatcctcattgtgagtggctggggatgcgtccagctgcctcgtggtgggggcccccagggtcctcattgtggtgggggcaggtctcaggatccctagggatttttcatttcttctcttccctctgagggacaagagcagggagcggggctggaagggtcagcttgagaccaaggctcacaggaggtgtgctcgcccctaggtgggctccagcctgtggaggacagtgcaggggagggtgaggagtgtaccggccccagcgtggctgagcacacagcctccaggccgaggacccagctgacagctttgcgcagtgatgataccctcgaggtggttgtgatgacatcagatttgcagaaaagaaaattgcttaagggccttgcccatgggcgcaaagctagtgaggaccatgttttccccctcctccatgccattgggacaccacagggtctgaatctggggcactaggggtggccccgttactgtgaaccacagcagtgaaatgtggaggccctgtagtcagttaacgtgaccagatacacataatggggagacgtcctgccgtgacttcatctcagagatttcgctgtcacgttagaggaggaggagcgtctgagccgtgcgcttggcatctgccccttagtgaaaaccctgggcatggcatgattaaggttgatgctccagtgtccagaaggttttctttttgcccacaagtatatcagggatgggatggtggacccaggctcctccaccaccagactgccttacctgagccctgctggccccaaagatatagaaggcaccctggttccctgtgctcacctggaccactgcctgcatcagctgggtcaggggaggatgggcagcccccacacctgcttcccaggggcaggttgcctggcggctctgattcccttggtgccagctgctgagaaccttactgccatttcagttgagcccacctagctctcatataaatacatgttccctgagggcatcttaccatcccatgtgaccactccagccagacaggggaggcagcacggcctcggggcacagcactgctccaggagtcaggaggcctgccttctggttcactcactaacaggtgaggtgatctaatgggggtgagaacttctgcccttaacacctcaagagctgttgcaggaccagggaagataatggggtgtctagcgccgttatccgactggtcctcgaacaagctcctgtgcccagggactagaccatgactcacagctcctgtccacaccagggatcaccacgctcaccctcccctccatgtcctgcagggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctgagggacatggtgtgccacaggctgcaagctgcagggaattttcattggatgcagttgtatagttttacactctagtgccatatatttttaagacttttctttccttaaaaaataaagtacgtgtttacttggtgaggaggaggcagaaccagctctttggtgccagctgtttcatcaccagactttggctcccgctttggggagcgcctcgcttcacggacaggaagcacagcaggtttatccagatgaactgagaaggtcagattagggcggggagaagagcatccggcatgagggctgagatgcgcaaagagtgtgctcgggagtggcccctggcacctgggtgctctggctggagaggaaaagccagttccctacgaggagtgttcccaatgctttgtccatgatgtccttgttattttattgcctttagaaactgagtcctgttcttgttacggcagtcacactgctgggaagtggcttaatagtaatatcaataaatagatgagtcctgttagaatcttggagtttggtccgttgtaaatgttgacccctctccctgcatcttgggcacccctgggataacttgtgctgtgagcccaggatggaggcagtttgccctgtttgaaggaacttttaatgatctcgcctctctgcacacatttctttaactagaaagtttcctaagcaaaggagttaggagagcagggtggcctgacatctgccagccctgagctgtaaggctgtggatgctgagcaggtccctggactcagttgtgcacggtggcacagacactgccaggtggttgccaaaacatccagtggttccttcagcaagtgttcaccctctgcagaagcctgtgagggcctgagctcagaaaccactctcctttccttctctggctttggccctgggcactgtggtgggagagtggacagtttggctttgccttctctgtacatcaatcatgggttgcaaagagaatctcagaagtgcctcttcctgagcacagtggctcacacctgtaatcccaatacttcgggaggtcgagtcggaaggatcacttgagcccaggagtttgagaccagccggggcaacatagtgagactttgtacaaaaaaaaatttaaaaattagccaagcatggtggcatgcatctgtagtctcagctactctggaggctgaggtgggaggatcacttaagcccaggaggctgaggctgcaatgagccgagatcaagcaggtgttaggtatatcagacagctgagaagacgcaagtgtgccctggggttcaaactggtacccctgtctccctgttccaggaataacatgagtgccgggacaatgcatctttattatgagaggaatgagaattgtgtatcttgacatttgacaggagcttgctttcccccaggctgtttgaggaagggcagaggaaaatgtggtgccctaagaaggaaggacagaggaggccgaacactggcgggtggaatcccactgattagtagtgcaggtcagagacctgggatggggggcattgccgtcatggaagccacagcggggagcgggtaaagcagacagggatggtccctgatggtgacaactcgcaagaggttaaggggaaagaaaaactgaaaagcttattcaatttggcaattatggcagtgtttatcttcagaagagcagttttagggtggggtttccaaagatgggattggacatatattttgaatcattaagcttgaggtctttcaaaggcctggccaagggttgctgggtggagaccacattcagaggtaaaggcagaaattgggggcccttaagtagacagcgagggaggaagaaatgaaggggcctggtgatggttagggtgaagtgttaagactgagaaaacaaggacatgtgagaagacgagggaagagcattggagagaacaaagacactggaggagatgctacttggaggtccccagagagcagggagacaaatgaacccagaacacaaatggcaaagaagaaaaatgagagaatttgtaaaagacagcattcgaacatgccgaacaagagcagggtactggtgttcaaacacctgtatctcccccgtgtaacccgtcaactaatatctttccatatttgctccagatttgtctttagaaataaaacccacgttctgaagtcctgtttgtatgtggccccagtcctgttgcctccgcctcctgtcctgaagtcgatttctgcccttctcatctatggttagttttgttttgtatgttggcatgttttcttaactttacagaaatggtatcatactgtacatatttgataattttttaaaatattgcattctggaggcatgtataaatgtagctccagttcatttattttatttattttttgagatggagttttgctcttgtcacccaggctagagtgcaatggcgtgatgttggctcactgcaacctctgcctcctgggttcaagcaattctcctgtctcaatttcctgagtagctgggattacagttgcccgccaccatgcctggctaattttgtattttagtagagacggggtttcaccacgttagccaggctggtctcaaactcctgactgcaggtgatccacgcaccttggcctccaaaagtgctgggattacaggcgtgagccaccgtgcccagcccagttattttaactattgtatagtgttccattgtatgagttctactgtttatatgctattgatcgacctgtaggggttttgcagtgtttctgtattacagctgtgctgcagtgagcatcccatcacattgtgtggatttgaggaagtattggaattcccccaattgactggacattcccaattaccctccaagtatgtgtctgtttatccttccatccgcaatctgagagttccccaactctataatacttggtgtcatcagacttttcatcttgtctgattggatgggtgtcatttcctttaggttttataattatcttttcatatgtgtattggctgtacaaggttccttctctgttcattattattaatttttttagacagagtctcgcgctgtcgcccaggctggagtgcagcagcgtgatcttggctcactgcaagctccgcctcccgggttcatgccattctcctgcctcagcctcctgagtagctgggattacaggtgcctgccatcacgcccggctagtttttttgtattttgagtagagatggggtttcaccgtgttagccaggagggtctcgatctcctgacctcgtgatccacccgcctcggcctcccaaagtgctgggattacaggtgtgagtcactgcgcccagcccaagtttccttctctgttacttgttcatatcctctgcccatttttcacttggattttttgtcttacggatatttaagcctcttaaaatatatattctggagagatgctaatctttgattaattatatgcattgcaaatgtctggtacattgtggcttgcctctcttccctgcctttaggagtgttttgctggacccaagtaatttttaaatgttaatgttattaaatctatcagttttttgcttgtatggcttatgccattgaatcttgttttaagagatccttccctaccctcaaggttttctaaatttttattttcataataagatttttagttcatctgaaatgtatttttatgattgtatttagtagggacctaattttgtttttctttgtaaccaggtgtcccagcactgtttactgaacagtctctcctttctcgctggtctgtagaactctcctgacatataccaagtttccataagtgggtggatgggttcctgagctctctactgttaatagaacttgctctctcgcaggccaatgcctcaccaggtgattgaagcagagaaacttaggtggtgaaaggagaagatggggcctgtcctgagagtttctgttcctgagatgctagaggcagagggtatgtaaatctgaagttacactggatctcctaaaacagtataaagctacagaagtataatagtgtggaatggtggtgggagtcagtaagggttaggtcactgcagtggtttaaacaagatgggctagaatcctttcacaggcacaggcagcttggagagggtgcaatagtgcatggtatcaggggtcagatgcctctttttcctttgagatcagtaagtggctttcacctcatgacctaggctggctgctgtgtgctagccgtcaagtcacactccatccagcatgaaaggaggttagaaaagggtgcatttcctcttcttaaaaacatgtctcaaagttgcacacagcacttttgcctatattcaattggccattagtcccacggccatacctgtctgagactgagagactgggaaatgtctttatttcaagtggccatatatccacctaaacaagataagggatacgtggttatggcgtgtcttttggtttaccaatgcagataatgaagttaccaaaacaatgagaaaatggggtcgtgagggatcatgtgaatcacaagctgatgtcttcaaagacggtggaaatgggccccgggaggcagcagatgacagcagtggggattaaggtagacctccatcctggggttaaaatgaggggaaggtgatggagctggaccagcagtcagaatggtcagtggttaggagaccctctgccccccaccgctgccaccattggctctctacagaatgcctgcgagtggcttagagtgaccaaggatgaggtgcagatccatgtgcacccccctgccccctctgtggacaattttcatgcctgacagcacagtctatgtggattgcaagccgatgaaactatgcaaagtagaagcatgcctgcagtttgtgattcggtgatgtgttttatgcttatgtgagtcgaatggggcggcagggtcctgtggtcacccgctgagaaggaagggtcctgtaaccactgcctttctttcagctacttgagaaaggtgttgtgagggaccgtggattttgggacagctttgaatggtggtagggaggaagggtccggtctgagtgaatggccagaaagctgtggggaagcttttaggacattggccaagagctccctgaaggcagccagggagatacttgtcagtacatgtgactaatggccaactgaatataagcagaagtgctgtgttgctgtgtgcaacactggacaccttaggaaggacctcgagacagtggttgtggactctgtagagagtaacagtgacagtagcaaacccttacccagtgccaaccttgtgctaggctcgcactaaatgagtttaccttcaattctcgtaacaataggaggtaactactattctaatttccattttatagatgaggaaactaaggcacagagatcactgacttgcccaaaatcaagcagggagtagttagtatataagcccacggtatgtggtttgtagaataggtgctcttgactagcagaaataggtcctccctgcagtgtgtaattgataacaagcatgggctgccatcttcctgtcgaggccactcaaaacacccaacaggctacgcacggtggctcacacctgtaatcccagcactgtgggaggccgaggtgggcgggtcacctgaggtcaggagttcgagaccagcctggccgacatggtgaaactccgtctctactaacagtacaaaaattagctgggcgtggtggcgggcacctgtaatcccagctactcaggaggctgagacagaagaatcacttgaaccagggaggcagaggttgcagtgagacaagatcacgccattgcactccagcctgtgtgacaaaagcgaaactgtctcaaaaaaaaaaaaaaaaaaagtatgattttataatcccagcaccttgggaggctgagtcgtgagaatcacttgagcccaggagtttaagaccaatctaggcaacatggcaagaccccatctctgccaaaaataaaaaatagtctaattttagctattcatgtgtgtgtgaagtggtgtctcttcgtggctttgatctgcatttccctaatgctgactaatgacgttgggcacctgttcatgtgcttactggtcagatatctttcttttgttacattttattaagttttaaaatttaaagtcaaagatttccctatgagaatgacttttaaaatgaccaaaaaggggaagataacattaattcttgaagagaaggcctctgagaaaaatacagttgtagcaagctgctactttgcaaatgacccatgcattttaattttcccctaaggaaggccaaggaagagtcttatcacctcagggcaggagatgtagggacttgggtcatttaataagagtggtaggtttgaaaactcaaacccagaagactccttagagtttctcccaggaggtagggaaggggccgcatccatggagagaggaggatgtgacttagagcagtggtccccaatctttagggaccagggactggtgtcatggtagacagtttttccacagataggggttggggggatgatttggagctgaaactgctccacctcaggtcatcaggcattagattctcatgtggagtgtgccacttagatccttggcgtgcacagttcacaatggggttcgaggtcctatgagaatccgatgccactgatttgacaggaggcggagctcaggtggtaatgctcatctccaccgcttaccacctgctgtgcagcctggttcctaataggctatagactggtactggtccatggcctgggggttggggacccctgatttagaggaagtaagggcatggcttaccgtgggccctggggtgttctgggaatggggaggatggagagaagagaggaggtagggaagacctccccttgctccccatttgggatttggggagaaagtcaggtctcaggctcaacagtacctgatcctgtaccatcttccaaagggaagtcagtggggttggaaggtaggcaggggttatcttctctgagccacggcacaagacagaagtttcccaccattcctgagggggcaggtggtaggtccccaagcagagagccagcagtccctctctgaggcctgcaatggaatggggtggggtgtccactgagccaagggtctgtcagtgagagctggggaggctgggctggcttgcaagcacctgttataaccaaaccaggaaatcaggttccgagtcttgccagcaagggcctacagctgccagcagagatggacagccaggagaccccaattggccacccagagccaccctcctctgcctaccccaccctccagtactccagagcctactcggaggggaacagaaacctgagaggctgaacacacacacatggagaaacaaacgtagtaaaatatttggggaatcaggaagaattatttgtactattcctgcaacctttctataggcttgaaattatcaaaataaatttttaaaaattgtaataacattctcatactaaaacactgagtttttttctttcattttttgattttttctttttgactccagcatgacttactctaacaatgggtggtctcgattttgaaatactttcttctccaagcctttcatgacaccctgtctctgttggttctgaaaatgttggattttgtctcagcccttgcttctggaaacagccaaggttaagaaaaccccccatgctttgtgttctagcagacagcttcctgcaaagagccatcttcccagagcacttaggcctcttagatgtctcccttgtttaattatgacaagagcacacacacagaccctccaaattcccattcttagtcttctaaatgattagctgagctgcttttccccactgattaatcggaataaaatgctcattaaccaaacttccctcctttccccaggtccctaaactttcctgagtcggcagacatcccctctggagaagaggttggccccagagtcgaacatcctctgatctacctgatcctgctgcccttccattccacttccccacatctgttctttctggtcgtgtttactcccctattaaaaaaacaaaaccagaaaacgtgtttgcctagatcttgagactctggaagatcttaacagtcagaggttccccctatttgcaatgatctcctttcctgccccttcctatccttgcaataatccttttgaataaagtctctccttactaaatccagttcctaaaaattaatttttttagag

SEQ ID NO: 2 - protein, 448 aa, Homo sapiens
  REGION 1..448: note = misc_feature - presenilin-2 isoform 1
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 3 - DNA, 19 bp, Homo sapiens
  misc_feature 1..19: note = Forward 5'-3' Primer
  source 1..19: mol_type = other DNA
catcagccctttgccttct

SEQ ID NO: 4 - DNA, 20 bp, Homo sapiens
  misc_feature 1..20: note = Reverse 3'-5' Primer
  source 1..20: mol_type = other DNA
ctcaccttgtagcagcggta

SEQ ID NO: 5 - DNA, 26 bp, Homo sapiens
  misc_feature 1..26: note = Forward 5'-3' Primer
  source 1..26: mol_type = other DNA
acagaattcgccccggcctggtacac

SEQ ID NO: 6 - DNA, 25 bp, Homo sapiens
  misc_feature 1..25: note = Reverse 3'-5' Primer
  source 1..25: mol_type = other DNA
taagcttggcacggctgtccaagga

SEQ ID NO: 7 - DNA, 20 bp, Homo sapiens
  misc_feature 1..20: note = Forward 5'-3' Primer
  source 1..20: mol_type = other DNA
tcagcatctacacgccattc

SEQ ID NO: 8 - DNA, 20 bp, Homo sapiens
  misc_feature 1..20: note = Reverse 3'-5' Primer
  source 1..20: mol_type = other DNA
agcaccaccaagaagatggt

SEQ ID NO: 9 - DNA, 26 bp, Homo sapiens
  misc_feature 1..26: note = Forwad 5'-3' Primer
  source 1..26: mol_type = other DNA
attcgccccggcctggtacactgcca

SEQ ID NO: 10 - DNA, 27 bp, Homo sapiens
  misc_feature 1..27: note = Reverse 3'-5' Primer
  source 1..27: mol_type = other DNA
ctgtccaaggagctgcaggcggcgcag

SEQ ID NO: 11 - DNA, 25 bp, Homo sapiens
  misc_feature 1..25: note = g1N141I guide RNA F
  source 1..25: mol_type = other DNA
caccgcatcatgatcagcgtcatcg

SEQ ID NO: 12 - DNA, 25 bp, Homo sapiens
  misc_feature 1..25: note = g1N141I guide RNA R
  source 1..25: mol_type = other DNA
aaaccgatgacgctgatcatgatgc

SEQ ID NO: 13 - DNA, 100 bp, Homo sapiens
  misc_feature 1..100: note = Donor ssODN#A N141I
  source 1..100: mol_type = other DNA
gagagaagcgtggctggagggcagggccagggcctcaccttgtagcagcggtacttgtagagcaccaccaagaagatggtcagggtgttcagcacggagt

SEQ ID NO: 14 - DNA, 21 bp, Homo sapiens
  misc_feature 1..21: note = Forward 5' Primer
  source 1..21: mol_type = other DNA
taacggcggcagacaaaaaga

SEQ ID NO: 15 - DNA, 22 bp, Homo sapiens
  misc_feature 1..22: note = Reverse 5'-3' Primer
  source 1..22: mol_type = other DNA
gaagtattgcttcagttggcct

SEQ ID NO: 16 - DNA, 21 bp, Homo sapiens
  misc_feature 1..21: note = Forward 5' Primer
  source 1..21: mol_type = other DNA
agaagaacggcaagtacgaga

SEQ ID NO: 17 - DNA, 21 bp, Homo sapiens
  misc_feature 1..21: note = Reverse 5'-3' Primer
  source 1..21: mol_type = other DNA
tgttgagggacagattgtggc

SEQ ID NO: 18 - DNA, 21 bp, Homo sapiens
  misc_feature 1..21: note = Forward 5' Primer
  source 1..21: mol_type = other DNA
taacggcggcagacaaaaaga

SEQ ID NO: 19 - DNA, 22 bp, Homo sapiens
  misc_feature 1..22: note = Reverse 5'-3' Primer
  source 1..22: mol_type = other DNA
gaagtattgcttcagttggcct

SEQ ID NO: 20-21 - skipped (2 entries)

SEQ ID NO: 22 - DNA, 20 bp, Homo sapiens
  misc_feature 1..20: note = Forward 5' Primer
  source 1..20: mol_type = other DNA
acgaatctccgaccaccact

SEQ ID NO: 23 - DNA, 20 bp, Homo sapiens
  misc_feature 1..20: note = Reverse 5'-3' Primer
  source 1..20: mol_type = other DNA
ccatggccacaacaactgac

SEQ ID NO: 24 - DNA, 22 bp, Homo sapiens
  misc_feature 1..22: note = Forward 5' Primer
  source 1..22: mol_type = other DNA
gaagtgtcccaggacatgataa

SEQ ID NO: 25 - DNA, 22 bp, Homo sapiens
  misc_feature 1..22: note = Reverse 5'-3' Primer
  source 1..22: mol_type = other DNA
ctcttgagtagctgggattgag

SEQ ID NO: 26 - DNA, 46 bp, Homo sapiens
  misc_feature 1..46: note = Forward 5'-3' Primer
  source 1..46: mol_type = other DNA
ctccgtgctgatcaccctcatcatgatcagcgtcatcggttatgac

SEQ ID NO: 27 - DNA, 48 bp, Homo sapiens
  misc_feature 1..48: note = Reverse 3'-5' Primer
  source 1..48: mol_type = other DNA
gaggcacgactagtgggagtagtactagtcgcagtagcaccaatactg

SEQ ID NO: 28 - DNA, 20 bp, Homo sapiens
  misc_feature 1..20: note = Forward 5'-3' Primer
  source 1..20: mol_type = other DNA
catcatgatcagcgtcatcg

SEQ ID NO: 29 - DNA, 25 bp, Homo sapiens
  misc_feature 1..25: note = g1N2411 guide RNA F
  source 1..25: mol_type = other DNA
caccgcatcatgatcagcgtcatcg

SEQ ID NO: 30 - DNA, 25 bp, Homo sapiens
  misc_feature 1..25: note = g1N2411 guide RNA R
  source 1..25: mol_type = other DNA
aaaccgatgacgctgatcatgatgc

SEQ ID NO: 31 - DNA, 118 bp, Homo sapiens
  misc_feature 1..118: note = Donor ssODN#A N141I
  source 1..118: mol_type = other DNA
gagagaagcgtggctggagggcagggccagggcctcaccttgtagcagcggtacttgtagagcaccaccaagaagatggtcataaccacgatgacgctgatcatgatgagggtgttca

(19) United States
(12) Patent Application Publication
(10) Pub. No.: US 2025/0257318 A1
(43) Pub. Date: Aug. 14, 2025

(54) METHOD AND COMPOSITION FOR GENERATING BASAL FOREBRAIN CHOLINERGIC NEURONS (BFCNS)

(71) Applicants: New York Stem Cell Foundation, Inc., New York, NY (US); Icahn School of Medicine at Mount Sinai, New York, NY (US)

(72) Inventors: Scott Noggle, New Rochelle, NY (US); Maitane Ortiz-Virumbrales, New York, NY (US); Sam Gandy, New York, NY (US); Ilya Kruglikov, New York, NY (US); Michelle Ehrlich, New York, NY (US)

(21) Appl. No.: 18/925,851

(22) Filed: Oct. 24, 2024

Related U.S. Application Data
(62) Division of application No. 17/146,336, filed on Jan. 11, 2021, now abandoned.
Application No. 17/146,336 is a continuation of application No. 15/990,208, filed on May 25, 2018, now Pat. No. 10,889,800.
(60) Provisional application No. 62/586,571, filed on Nov. 15, 2017.
(60) Provisional application No. 62/574,639, filed on Oct. 19, 2017.
(60) Provisional application No. 62/571,741, filed on Oct. 12, 2017.
(60) Provisional application No. 62/511,271, filed on May 25, 2017.

(51) Int. Cl. C12N 5/0793; A61K 35/30; C12N 5/074; C12N 9/22; C12N 15/11; G01N 33/50

(52) U.S. Cl. CPC C12N 5/0619; A61K 35/30; C12N 5/0696; C12N 9/22; C12N 15/11; G01N 33/5058; C12N 2310/20; C12N 2501/15; C12N 2501/41; C12N 2501/999; C12N 2506/02; C12N 2506/45; C12N 2510/00; C12N 2800/80; G01N 2800/2814

(57) ABSTRACT

The invention relates to methods and compositions for developing basal forebrain cholinergic neurons (BFCNs) from stem cells, and in particular, BFCNs having repaired electrophysiological defects relating to one or more mutations in PSEN2, and to the use of such BFCNs in cell-based therapies to treat Alzheimer's disease.

### CROSS REFERENCE TO RELATED APPLICATIONS

This application is a continuation application of U.S. application Ser. No. 15/990,208 filed May 25, 2018, now issued as U.S. Pat. No. 10,889,800; which claims the benefit under 35 USC § 119(e) to U.S. Application Ser. No. 62/586,571 filed Nov. 15, 2017, U.S. Application Ser. No. 62/574,639 filed Oct. 19, 2017, U.S. Application Ser. No. 62/571,741 filed Oct. 12, 2017 and U.S. Application Ser. No. 62/511,271 filed May 25, 2017, all now expired. The disclosure of each of the prior applications is considered part of and is incorporated by reference in the disclosure of this application.

### STATEMENT REGARDING FEDERALLY SPONSORED RESEARCH OR DEVELOPMENT

This invention was made with government support under Grant Nos. AG005138, AG046170 and AG042965 awarded by the National Institutes of Health. The government has certain rights in this invention.

### INCORPORATION OF SEQUENCE LISTING

The material in the accompanying sequence listing is hereby incorporated by reference into this application. The accompanying sequence listing text file, named NYSC1390-5_ST25.txt, was created on Jan. 8, 2021 and is 53 kB in size. The file can be accessed using Microsoft Word on a computer that uses Windows OS.

### BACKGROUND OF THE INVENTION

### Field of the Invention

The present invention relates generally to the field of medicine, and more specifically to methods and compositions for developing basal forebrain cholinergic neurons (BFCNs) from stem cells, and in particular, BFCNs comprising repaired electrophysiological defects relating to one or more mutations in the presenilin 2 gene (PSEN2), and the use of such BFCNs in cell-based therapies to treat Alzheimer's disease.

### Background Information

Alzheimer's disease (AD) is a progressive disease resulting in senile dementia. Broadly speaking the disease falls into two categories: late onset, which occurs in old age (65+ years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, senile plaques and neurofibrillary tangles. Senile plaques are areas of disorganized neuropil up to 150 m across with extracellular amyloid deposits at the center visible by microscopic analysis of sections of brain tissue. Neurofibrillary tangles are intracellular deposits of microtubule associated tau protein consisting of two filaments twisted about each other in pairs.

The principal constituent of the plaques is a peptide termed Aβ or β-amyloid peptide. Aβ peptide is an internal fragment of 39-43 amino acids of a precursor protein termed amyloid precursor protein (APP). Several mutations within the APP protein have been correlated with the presence of Alzheimer's disease. Such mutations are thought to cause Alzheimer's disease by increased or altered processing of APP to Aβ, particularly processing of APP to increased amounts of the long form of Aβ (i.e., Aβ1-42 and Aβ1-43). Mutations in other genes, such as the presenilin genes, PSEN1 and PSEN2, are thought indirectly to affect processing of APP to generate increased amounts of long form Aβ. These observations suggest that Aβ, and particularly its long form, is a causative element in Alzheimer's disease.

There are 5 million people currently affected by Alzheimer's disease in the US and, according to the Alzheimer's Association, this number will increase to 16 million by the year 2050. Unfortunately, we only have direct evidence for genetic causation that accounts for 3-5% of these patients. This percentage encompasses the autosomal dominant early onset familial Alzheimer's disease (EOFAD) variants caused by inherited fully penetrant autosomal dominant mutations in the APP, or PSEN1, PSEN2 that constitute the γ-secretase apparatus [87], and changes in their function increases the production of Aβ42 oligomers and/or deposition of amyloid plaques.

After decades studying murine models of AD that do not fully recapitulate the pathophysiology of this disease in the human brain [5, 57, 58], a complementary new concept of AD modeling in vitro has emerged upon the breakthrough by [81] allowing adult human tissue reprogramming into iPSC using defined factors, and their subsequent in vitro differentiation into specific brain cell types.

BFCNs are one of the most vulnerable neuronal populations whose deterioration explains, in part, the cognitive decline in AD patients. Apart from the evidence for BFCN failure and atrophy, other studies have revealed that human embryonic stem cell-derived BFCNs transplanted into AD mouse models can be associated with improvement in the learning behavior of the implanted mouse [94]. These findings highlight the relevance of iPSC- and ESC-derived BFCNs as not only early clinical indicators but also as a potential strategy for subtype-specific cell-based therapy for AD [39]. In order to move this cell-based therapeutic strategy forward, there has been an urgent need for a refined differentiation protocol to generate human ESC- and/or iPSC-derived BFCNs.

### SUMMARY OF THE INVENTION

The present invention provides a highly reproducible protocol to efficiently derive BFCNs from pluripotent stem cells (PSCs), including embryonic stem cells (ESCs) and induced pluripotent stem cells (iPSCs).

Accordingly, in one embodiment, the invention provides a method of generating BFCNs. The method includes culturing PSCs in a basal media comprising an inhibitor of transforming growth factor beta (TGF-β) signaling and an activator of sonic hedgehog (Shh) signaling to induce neuroectodermal differentiation. In some aspects, the basal media is a modified mTeSR1 formulation that lacks factors that support pluripotency including basic fibroblast growth factor (bFGF), TGF-β, lithium chloride (Li—Cl), GABA and pipecolic acid. In some aspects, culturing is performed in the presence of dual SMAD inhibitors, such as SB431542 and LDN193189 along with one or more agonists of smoothened protein, such as smoothened agonist (SAG) and purmorphamine. After about 9, 10, 11 or 12 days of culturing, CD271+ cells are selected and in a neuronal basal medium, such as Brainphys™ to generate neuronal embryoid bodies (NEBs). The neuronal basal medium is optionally supplemented with one or more of B27 supplement, an inhibitor of rho-associated protein kinase (ROCK), nerve growth factor (NGF) and brain derived neurotrophic factor (BDNF). After about 7, 8, 9 or 10 days of culturing the CD271+ cells, the formed NEBs are harvested, dissociated, and plated as monolayer cultures and further cultured in a neuronal basal medium optionally supplemented with B27 supplement, NGF and BDNF. To ensure differentiation into BFCNs, the cultured cells are analyzed for positive expression of Tuj1, MAP2, BF1, Nkx2.1 and p75.

In another embodiment, the method utilizes iPSCs which may be treated with a gene editing system to repair one or more mutations, such as a mutation of presenilin 1 (PSEN1) or presenilin 2 (PSEN2). In one aspect, the mutation is PSEN2^{N141I}, repair of which restores neuronal excitability in BFCNs.

Accordingly, in another embodiment, the invention provides a method of treating a disease or disorder in a subject. The method includes administering to a subject a BFCN generated using the culturing method described herein. In certain aspects, the disease or disorder is an amyloidogenic disease, such as systemic amyloidosis, Alzheimer's disease, mature onset diabetes, Parkinson's disease, Huntington's disease, fronto-temporal dementia, and prion-related transmissible spongiform encephalopathies. In embodiments, a BFCN having a mutation that impairs neuronal excitability, such as PSEN2^{N141I}, may be obtained from a subject and used to generate an iPSC, which in turn may be treated with a gene editing system to repair the mutation. The gene edited iPSC is then cultured as described herein to produce BFCNs having restored neuronal excitability which are administered to the subject to treat the disease or disorder. In certain aspects, the disease or disorder is AD.

In a related embodiment, the invention provides a method of restoring neuronal excitability in BFCNs in a subject. The method includes: a) isolating a BFCN from the subject, wherein the BFCN has a mutation in PSEN2 resulting in impaired neuronal excitability of the BFCN; b) generating an iPSC using the BFCN of (a); c) repairing the PSEN2 mutation in the iPSC; d) culturing the iPSC of (c) using the differentiation protocol described herein to generate a BFCN having the repaired mutation; and e) administering the iPSC of (d) to the subject, thereby restoring neuronal excitability in BFCNs in the subject.

Also provided is a method of identifying a compound for treatment or prevention of a disease or disorder associated with diminished neuronal excitability in BFCNs. The method includes: a) contacting a BFCN or neuronal embryoid body (NEB) generated using the differentiation protocol described herein with a candidate compound, wherein the BFCN comprises a mutation in PSEN2 resulting in impaired neuronal excitability of the BFCN; and b) detecting neuronal excitability of the BFCN after contact with the candidate compound. An increase in neuronal excitability of the BFCN after contact with the candidate compound identifies the compound as a compound potentially capable of restoring neuronal excitability in BFCNs.

The invention further provides a BFCN generated using the differentiation protocol as described herein. The BFCN may include a gene edited repair of PSEN2, as well as a detectable marker recombinantly introduced into the BFCN genome.

The invention also provides a kit for generating a BFCN. The kit includes a culture media having an inhibitor of TGF-β signaling and an activator of Shh signaling. In embodiments, the culture media is a modified mTeSR1 formulation that lacks factors that support pluripotency including bFGF, TGF-β, lithium chloride (Li—Cl), GABA and pipecolic acid. In embodiments, the culture media includes dual SMAD inhibitors, such as SB431542 and LDN193189, along with one or more agonists of smoothened protein, such as smoothened agonist (SAG) and purmorphamine. The kit may also include a neuronal basal medium, such as Brainphys™ optionally supplemented with one or more of B27 supplement, an inhibitor of ROCK, NGF and BDNF. In embodiments, the kit includes reagents for detection of CD271+ cells as well as cells which positively express Tuj1, MAP2, BF1, Nkx2.1 and p75.

### BRIEF DESCRIPTION OF THE DRAWINGS

FIGS. 1A-1E. Overview schematic of basal cholinergic differentiation protocol. (FIG. 1A) Cells are plated and allowed to reach 100% confluency (Day 0), before the initiation of dual SMAD inhibition and the subsequent introduction of ventralizing agents (Day 2). At day 10 the monolayer is dissociated, sorted for p75+ cells, and kept as NEBs until day 19. Then the culture is dissociated again into a monolayer (See Methods for more details). (FIG. 1B) Left panel shows sustained EGFP expression driven by Nkx2.1 induction in NKx2.1-EGFP hESCs upon SHH plus purmorphamine or SAG plus purmorphamine treatment, maintained at Day 14, after removal of treatment at Day 8. Right panel shows Nkx2.1, Lhx8 and BF1 relative gene expression to GAPDH measured by qPCR, in NKx2.1-EGFP cell line in the presence of the indicated ventralizing agents, or unpatterned (UNP) at Day 12. n=3, in technical triplicates. (FIG. 1C) Confocal microscope images of Nestin, Sox2 and DRAQ5 immunostaining in fControl and control lines at Day 11, showing typical neural rosettes (left panel), or Tuj1, Nkx2.1 right and DRAQ5 in the right panel. Images representative of 3 independent experiments. (FIG. 1D) Fluorescence microscope images of immunostained NEB cryosections or dissociated NEBs into a monolayer with the BFCN markers Nkx2.1/Tuj1/p75/BF1/MAP2/ChAT. (FIG. 1E) Dissociated NEBs into a monolayer immunostained at Day 50 with MAP2, ChAT and Hoescht. Fluorescence microscope images the effect of NGF addition to SAG plus purmorphamine treatment alone. Images are representative of at least 3 independent experiments.

FIGS. 2A-2F. Basal cholinergic markers in PSEN2^{N141I}, neuroprecursors. (FIG. 2A) Table showing the cell lines used. Four iPS lines reprogrammed from fibroblasts were used; two controls (949 and 050643, labelled as fControl and Control, respectively) that do not carry the PSEN2^{N141I} mutation nor the £4 allele; and two AD patients (948 and 950, labelled as AD1 and AD2, respectively) who carry the mutation and the £4 allele. Three of the four iPS lines were family related (fControl, AD1, and AD2). (FIG. 2B) Representative Sanger sequencing chromatograms showing a fragment of exon 5 of PSEN2. Arrow marks site of the missense point mutation Chr1:227,073,304 A>T. (FIG. 2C) Immunocytochemistry and RT-PCR for early neuronal and basal forebrain markers. n 3, 3 independent experiments with technical triplicates. (FIG. 2D) RTPCR fold changes for TUJ1 and BF1. n 3, 3 independent experiments with technical triplicates. (FIG. 2E) Representative histograms for P75 staining. n>6. (F) Aβ40 and Aβ42 ELISA quantifications. n 3, 3 independent experiments with technical triplicates. ***, p<0.001. *, p<0.05.

FIGS. 3A-3B. Neuronal and basal cholinergic markers by immunocytochemistry. (FIG. 3A) Immunostaining for TrkA on DIV 21. (FIG. 3B) Immunostainings for ChAT and vAChT at different magnifications at DIV65; and Tuj1 and MAP2. Images are representative of at least 3 independent experiments.

FIGS. 4A-4D. CRISPR/Cas9-mediated correction of PSEN2^{N141I} J iPS lines. (FIG. 4A) Schematic showing guide RNAs used in the targeting of CRISPR/Cas9, as well as donor ssODNs utilized to introduce wild-type genotype. Sequence identifiers from top to bottom: SEQ ID NOs: 26-31. (FIG. 4B) Left 2 panels show GFP positive HEK293T cells indicating Cas9 system with guide RNA expression, NT refers to non-transfected; right 2 panels show sample of GFP positive iPSCs after lipofection with pCas9-gN141I-GFP vector. (FIG. 4C) Sanger sequencing results from iPSC lines, showing corrections in the N141I mutation. (FIG. 4D) Aβ 42/40 ratio detected by ELISA in 72 h conditioned media from mutant, control or Cispr-Cas9 corrected BFCNs (DIV 34). n 4, 4 independent experiments with technical triplicates. *, p<0.05; **, p<0.01 Student T-test.

FIGS. 5A-5B. BFCNs carrying various PSEN mutations are not consistently more susceptible to Aβ42 oligomer toxicity. (FIG. 5A) Sample images of BFCNs from the indicated genotypes treated with propidium iodide to visualize cell death in response to 72-h exposure to Aβ42 oligomers (5 μM). (FIG. 5B) % LDH Release recorded from media collected after 72-h exposure. n 3, 3 independent experiments with technical triplicates. *, p<0.05; **, p<0.01 as detected by 2-Way ANOVA Bonferroni post hoc tests.

FIGS. 6A-6F. NLRP2 inflammasome mRNA levels are over-expressed in some PSEN2^{N141I} cells, but it is not driven by mutation. RT-PCR expression of (FIG. 6A) NLRP2, (FIG. 6B) NLRP3, and (FIG. 6C) ASB9 in cholinergic neuroprecursors. (FIG. 6D) Western blot showing NLRP2, PSEN2 and β-Actin. RT-PCR expression of NLRP2 in Neuroprecursors (FIG. 6E) and BFCNs (F). n 3, 3 independent experiments with technical triplicates, for all panels. ***, p<0.001.

FIGS. 7A-7B. Electrophysiological and morphological features of BFCN. (FIG. 7A) Top row—compound sodium and potassium currents produced by a voltage protocol shown in bottom row. Current trace produced by a voltage step to −20 mV shown in red. Inset shows first 25 ms of a current produced by a voltage step to −20 mV (scale bars 200 pA, 5 ms). (FIG. 7B) Differential interference contrast image of a patched BFCN recorded in (A). Ninety-four neurons (22 wild-type control, 21 familial control, 18 AD1, 28 AD2 and 5 iAD1_control). Scale bar is 30 μm.

FIGS. 8A-8C. Electrophysiological deficits in BFCNs from AD lines. (FIG. 8A) Co-localization of biocytin-labelled neurons with cholinergic markers ChAT and VAChT. Arrows indicate positions of recorded neurons somas, scale bar is 50 μm. (FIG. 8B) Representative firing patterns of BFCNs produced by a 1 sec negative and positive square current injection are depicted. A grand total of 94 individual neurons were studied electrophysiologically: 22 wild-type control neurons, 21 familial control neurons, 18 AD1 neurons, 28 AD2 neurons, and 5 iAD1_(CRISPR-corrected) neurons. The experiments on the 94 neurons required days to weeks. On each experimental day, representatives from each genotype were included, with at least three samples from each genotype studied each day. (FIG. 8C) Summary data on maximum number of action potentials that neurons are capable of sustaining (left) and height of a single action potential at rheobase (right) across all conditions. Individual data points are shown as circles, group means are shown as bars. **, p<0.01 Tukey HSD test.

FIGS. 9A-9D. Intrinsic electrophysiological properties of BFCNs. Summary data on all recorded BFCNs from five groups. Ninety-four neurons (22 wild-type control, 21 familial control, 18 AD1, 28 AD2 and 5 iAD1_control). Histograms show individual values from each neuron (circle) and group means (bars) for membrane resistance (FIG. 9A), capacitance (FIG. 9B), resting potential (FIG. 9C) and rheobase current (FIG. 9D). Statistical significance was tested with ANOVA and Tukey's post hoc comparisons.

FIGS. 10A-10B. Quality control of iPSC lines. (FIG. 10A) Immunofluorescence shows expression of pluripotency markers SSEA4, Nanog, Tra160 and in 7889(S)B iPSC line. (FIG. 10B) Three germ layers (endoderm, mesoderm, and ectoderm) from teratomas generated by 7889(S)B iPSC line.

FIGS. 11A-11B. Amyloid 3 levels in mature BFCNs. (FIG. 11A) Levels of Aβ40 on BFCNs (DIV 34). *, P<0.01 vs. other lines in study according to One-Way ANOVA Bonferroni Post-hoc test. (FIG. 11B) Levels of Aβ42 on BFCNs (DIV 34). n=3, 3 independent experiments with technical triplicates. *, P<0.01 based on Student's T-test.

### DETAILED DESCRIPTION OF THE INVENTION

The present invention is based on the discovery of a robust, fast, and reproducible differentiation protocol to generate BFCNs from PSCs using a chemically defined medium.

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned herein are expressly incorporated by reference in their entirety.

Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

Unless otherwise defined, all technical and scientific terms used herein have the same meaning as commonly understood by one of ordinary skill in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al. (2000, Molecular Cloning—A Laboratory Manual, Third Edition, Cold Spring Harbor Laboratories); and Ausubel et al. (1994, Current Protocols in Molecular Biology, John Wiley & Sons, New-York).

The terminology used in the description is for describing particular embodiments only and is not intended to be limiting of the invention. Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements).

It should also be understood that, in certain methods described herein that include more than one step or act, the order of the steps or acts of the method is not necessarily limited to the order in which the steps or acts of the method are recited unless the context indicates otherwise.

The term “PSEN2 gene” refers herein to a gene that encodes a PSEN2 polypeptide. The PSEN2 gene is represented by NCBI Reference Sequence: NC_000001.11 (SEQ ID NO: 1) as well as known orthologs. The term “PSEN2 polypeptide” refers herein to a polypeptide that is represented by NCBI Reference Sequence: NP_000438.2 (SEQ ID NO: 2) as well as known orthologs.

The term “amyloidogenic disease” includes any disease associated with (or caused by) the formation or deposition of insoluble amyloid fibrils. Exemplary amyloidogenic diseases include, but are not limited to systemic amyloidosis, Alzheimer's disease, mature onset diabetes, Parkinson's disease, Huntington's disease, fronto-temporal dementia, and the prion-related transmissible spongiform encephalopathies (kuru and Creutzfeldt-Jacob disease in humans and scrapie and BSE in sheep and cattle, respectively). Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient. The terms “β-amyloid protein”, “β-amyloid peptide”, “β-amyloid”, “Aβ” and “Aβ peptide” are used interchangeably herein.

The term “patient” or “subject” is used throughout the specification to describe an animal, preferably a human or a domesticated animal, to whom treatment, including prophylactic treatment, with the compositions according to the present disclosure is provided. For treatment of conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The terms “β-amyloid protein”, “β-amyloid peptide”, “β-amyloid”, “Aβ” and “AD peptide” are used interchangeably herein. Aβ peptide (e.g., Aβ 39, Aβ 40, Aβ 41, Aβ 42 and Aβ 43) is about 4-kDa internal fragment of 39-43 amino acids of APP. Aβ 40, for example, consists of residues 672-711 of APP and Aβ 42 consists of residues 672-713 of APP. Aβ peptides include peptides resulting from secretase cleavage of APP and synthetic peptides having the same or essentially the same sequence as the cleavage products. Aβ peptides can be derived from a variety of sources, for example, tissues, cell lines, or body fluids (e.g., sera or cerebrospinal fluid). For example, an A β can be derived from APP-expressing cells such as Chinese hamster ovary (CHO) cells stably transfected with APP_{717V} F, as described, for example, in Walsh et al. (2002), Nature, 416, pp 535-539. An A B preparation can be derived from tissue sources using methods previously described (see, e.g., Johnson-Wood et al. (1997), Proc. Natl. Acad. Sci. USA 94:1550). Alternatively, Aβ peptides can be synthesized using methods which are well known to those in the art. See, for example, Fields et al., Synthetic Peptides: A User's Guide, ed. Grant, W. H. Freeman & Co., New York, N.Y., 1992, p 77). Hence, peptides can be synthesized using the automated Merrifield techniques of solid phase synthesis with the .alpha.-amino group protected by either t-Boc or F-moc chemistry using side chain protected amino acids on, for example, an Applied Biosystems Peptide Synthesizer Model 430A or 431. Longer peptide antigens can be synthesized using well known recombinant DNA techniques. For example, a polynucleotide encoding the peptide or fusion peptide can be synthesized or molecularly cloned and inserted in a suitable expression vector for the transfection and heterologous expression by a suitable host cell. A B peptide also refers to related Aβ sequences that results from mutations in the Aβ region of the normal gene.

As used herein the phrase “substantially pure” refers to a population of cells wherein at least 95% of the cells have the recited phenotype. In all embodiments that refer to a “substantially pure” cell population, alternative embodiments in which the cell populations have a lower or higher level of purity are also contemplated. For example, in some embodiments, instead of a given cell population being “substantially pure” the cell population may be one in which at least 35%, 40%, 45%, 50%, 55%, 60%, 65%, 70%, 75%, 80%, 85%, 90%, 95%, 96%, 97%, 98%, or 99% of the cells, or 100% of the cells, have the recited phenotype.

The terms “co-administration”, “co-administered” and “co-administering” or “combination therapy” refer to both concurrent administration (administration of two or more agents at the same time) and time varied administration (administration of one or more agents at a time different from that of the administration of an additional agent or agents), as long as the agents are present in the area to be treated to some extent, preferably at effective amounts, at the same time.

The term “therapeutically effective amount” means the amount required to achieve a therapeutic effect. The therapeutic effect could be any therapeutic effect ranging from prevention, symptom amelioration, symptom treatment, to disease termination or cure, e.g., the treatment of Alzheimer's disease or an associated condition.

As used herein, the term “administering” is meant to refer to a means of providing the composition to the subject in a manner that results in the composition being inside the subject's body. Such an administration can be by any route including, without limitation, subcutaneous, intradermal, intravenous, intra-arterial, intraperitoneal, and intramuscular.

The term “effective” is used to describe an amount of a compound, composition or component which, when used within the context of its intended use, effects an intended result. The term effective subsumes all other effective amount or effective concentration terms, which are otherwise described or used in the present application.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

As used herein, “kits” are understood to contain at least the non-standard laboratory reagents of the invention and one or more non-standard laboratory reagents for use in the methods of the invention.

The term “obtaining” is understood herein as manufacturing, purchasing, or otherwise coming into possession of.

As used herein, the term “treated,” “treating” or “treatment” includes the diminishment or alleviation of at least one symptom associated or caused by the state, disorder or disease being treated. A subject that has been treated can exhibit a partial or total alleviation of symptoms (for example, Alzheimer's disease or associated condition), or symptoms can remain static following treatment according to the invention. The term “treatment” is intended to encompass prophylaxis, therapy and cure.

As used herein, the term “control” refers to a sample or standard used for comparison with an experimental sample. In some embodiments, the control is a sample obtained from a healthy patient. In other embodiments, the control is a historical control or standard reference value or range of values (such as a previously tested sample, subject, or group of samples or subjects).

### Methods

BFCNs are believed to be one of the first cell types to be affected in all forms of AD, and their dysfunction is clinically correlated with impaired short-term memory formation and retrieval. As detailed in the Example of this disclosure, the inventors present an optimized in vitro protocol to generate human BFCNs from iPSCs, using cell lines from PSEN2 mutation carriers and controls. Cell lines harboring the PSEN2^{N141I} mutation displayed an increase in the Aβ42/40 in iPSC-derived BFCNs. Neurons derived from PSEN2^{N141I}, lines generated fewer maximum number of spikes in response to a square depolarizing current injection. The height of the first action potential at rheobase current injection was also significantly decreased in PSEN2^{N141I} BFCNs. CRISPR/Cas9 correction of the PSEN2 point mutation abolished the electrophysiological deficit, restoring both the maximal number of spikes and spike height to the levels recorded in controls. Increased Aβ42/40 was also normalized following CRISPR/Cas-mediated correction of the PSEN2^{N141I} mutation. The genome editing data set forth herein confirms the robust consistency of mutation-related changes in Aβ42/40 ratio while also showing a PSEN2-mutation-related alteration in electrophysiology.

Accordingly, in one embodiment, the invention provides a method of generating BFCNs. The method may first include preparing PSC colonies. PSCs are seeded (plated) at low density and grown in an adherent culture for about 1-2 days. “Low density” means about 8,000 to about 11,000 cells/cm². Cells are preferably seeded at about 9,500 to about 10,500 cells/cm², more preferably at about 10,000 cells/cm². After about 1-2 days (or greater, i.e., 3, 4, 5, 6, 7, 8, 9, 10 or more), the PSCs form colonies, which are preferably about 75 μm to about 300 μm in diameter, more preferably about 100 μm to about 250 μm in diameter.

The term “PSCs” has its usual meaning in the art, i.e., self-replicating cells that have the ability to develop into endoderm, ectoderm, and mesoderm cells. Preferably, PSCs are hPSCs. PSCs include ESCs and iPSCs, preferably hESCs and hiPSCs. PSCs can be seeded on a surface comprising a matrix, such as a gel or basement membrane matrix. A preferable matrix is the protein mixture secreted by Engelbreth-Holm-Swarm (EHS) mouse sarcoma cells, sold under trade names including MATRIGEL®, CULTREX®, and GELTREX®. Other suitable matrices include, without limitation, collagen, fibronectin, gelatin, laminin, poly-lysine, vitronectin, and combinations thereof.

In some embodiments, media suitable for use in maintaining pluripotent stem cells is used. In embodiments such a medium is mTeSR1 medium from Stem Cell Technologies. However, one of skill in the art will recognize that there are several other types of media that are equivalent to mTeSR medium in terms of their suitability for use in maintaining pluripotent stem cells, any of which could be used. Typically such media will contain one or more pluripotency factors to facilitate the maintenance of cells in a pluripotent state. The composition of mTeSR1 medium is known in the art and described in, for example, Ludwig et al., 2006 (Nat Methods. 2006 August; 3(8):637-46; “Feeder-Independent Culture of Human Embryonic Stem Cells”), the contents of which are hereby incorporated by reference.

The pluripotent stem cells used in the method of the invention can be any suitable type of pluripotent stem cells. Where iPSCs are used, such cells may have been “reprogrammed” to the pluripotent state from a non-pluripotent state using any suitable means known in the art, including, but not limited to, modified RNA-based methods, Sendai virus based methods, and the like. Furthermore, such cells may have been reprogrammed to the pluripotent state using any suitable cocktail of reprogramming factors known in the art.

In one embodiment, after PSCs are prepared and grown to confluence in, for example mTeSR1 media, the method includes culturing the PSCs in a basal media comprising an inhibitor of transforming growth factor beta (TGF-β) signaling and an activator of sonic hedgehog (Shh) signaling to induce neuroectodermal differentiation. The basal media utilized is a modified mTeSR1 medium, which is a variant of mTeSR1 medium (sometimes referred to herein as “mTeSR1 Custom” medium) that does not comprise lithium chloride, GABA, pipecolic acid, bFGF or TGFβ1. Inhibitors of TGFβ signaling include, for example, one or more of SB431542, GW788388, LDN193189, LY2109761, LY2157299, and LY364947. Activators of Shh signaling include agonists of Smoothened, such as Smoothened Agonist (SAG; 3-chloro-N-[(1r,4r)-4-(methylamino)cyclohexyl]-N-[3-(pyridin-4-yl)benzyl]benzo[b]thiophene-2-carboxamide) and purmorphamine.

After about 6, 7, 8, 9, 10, 11 or 12 days (or greater, i.e., 15, 16, 17, 18, 19, 20, 25, 30 or more) of culturing, CD271+ cells are selected and cultured in a neuronal basal medium, such as Brainphys™ to generate neuronal embryoid bodies (NEBs). The neuronal basal medium is optionally supplemented with one or more of B27 supplement, an inhibitor of rho-associated protein kinase (ROCK), nerve growth factor (NGF) and brain derived neurotrophic factor (BDNF). Inhibitors of ROCK include, for example, GSK269962, GSK429286, H-1152, HA-1077, RKI-1447, thiazovivin, Y-27632, or derivatives thereof.

To select for CD271+ cells, overconfluent cells are lifted from the culture surface and purified by FACS and re-plated. This process allows for the formation of cell aggregates or spheres, also referred to herein as NEBs. For purposes of the present invention, the terms “NEB,” “aggregate” and “sphere” are used interchangeably and refer to a multicellular three-dimensional structure, preferably, but not necessarily, of at least about 100 cells.

Lifting can be performed mechanically, with a cell scraper or other suitable implement, or chemically. Chemical lifting can be achieved using a proteolytic enzyme, for example, collagenase, trypsin, trypsin-like proteinase, recombinant enzymes, such as that sold under the trade name TRYPLE™, naturally derived enzymes, such as that sold under the trade name ACCUTASE™, and combinations thereof. Chemical lifting can also be done using a chelator, such as EDTA, or a compound such as urea. Mechanical lifting or detachment offers the advantage of minimal cell death, however it produces aggregates of variable size, thus suitable spheres need to be selected through a manual picking process. Good spheres are defined as those having a round-shape, golden/brown color, with darker core and with a diameter between about 300 μm and about 800 μm. Detaching the cells using chemical methods, such as enzymatic digestion predominantly produces spheres that are appropriate for further culture. Therefore manual picking of spheres is not required, and the detachment steps can be adapted for automation and used in high throughput studies. However, enzymatic digestion increases cell death, resulting in a lower number of spheres.

After about 5, 6, 7, 8, 9 or 10 days (or greater, i.e., 11, 12, 13, 14, 15, 16, 17, 18, 19, 20, 25, 30 or more) of culturing selected CD271+ cells, the formed NEBs are harvested, dissociated, and plated as monolayer cultures and further cultured in a neuronal basal medium, such as Brainphys™, optionally supplemented with B27 supplement, NGF and BDNF. The surface on which the cells are plated and cultured can comprise an extracellular matrix protein (e.g., collagen, fibronectin, laminin) and/or a positively charged poly-amino acid (e.g., poly-arginine, poly-lysine, poly-ornithine). Preferably the surface comprises laminin and/or poly-omithine.

To ensure differentiation into BFCNs, the cultured cells are analyzed for positive expression of Tuj1, MAP2, BF1, Nkx2.1 and p75.

Many of the embodiments of the present invention involve certain factors to be used in (or excluded from) the compositions and methods described herein, for example as media supplements. These include, but are not limited to, bFGF, GABA, pipecolic acid, lithium chloride, TGF-β, NGF and BDNF. Each of these factors is well known in the art, including the full names of each of these factors in the cases where acronyms or other abbreviations are used. Furthermore, all of these factors are available to the public from multiple sources, including commercial sources. Exemplary amounts/concentrations for use of each of these factors in the methods and compositions of the present invention are provided in the Examples section of this patent disclosure. For all embodiments where specified amounts are referred to, amounts that are “about” the specified amount are also intended. Furthermore, one of skill in the art will recognize that in some situations further deviations from the specified amounts can be used, and will be able to determine how much of each factor to use by performing routine testing, optimization, dose-response studies, and the like, for example to reduce or increase the specified amounts, as long as the amounts used still achieve the stated effect, e.g., the stated differentiation effect. For example, in some embodiments specified amounts of the specified agents may be reduced to 10%, or 20%, or 30%, or 40%, or 50%, or 60%, or 70%, or 80%, or 90% of the stated amounts. Similarly, in some embodiments the specified amounts of the specified agents may be increased by 10%, by 20%, by 30%, by 40%, by 50%, by 60%, by 70%, by 80%, by 90%, by 100%, by 150%, by 200%, by 300%, by 400%, or by 500% of the stated amounts. Similarly, where specified factors are referred to, one of skill in the art will recognize that analogs, variants, or derivatives of such factors can also be used as long as the analogs, variants, or derivatives have the same general function/activity as the specified factors.

As discussed herein, the inventors have discovered that mutation of PSEN2 results in impaired neuronal excitability in BFCNs. As discussed in the Example herein the inventors observed significant mutation-related, editing-reversible differences in excitability of BFCNs via repair of PSEN2^{N141I} mutation which restored neuronal excitability. Accordingly, the method of the invention may utilize iPSCs which may be treated with a gene editing system to repair one or more mutations, such as a mutation of presenilin 1 (PSEN1) or presenilin 2 (PSEN2). In one embodiment, the mutation is PSEN2^{N141I}, repair of which restores neuronal excitability in BFCNs.

As used herein the term “gene editing” or “genome editing” refers to a type of genetic engineering in which DNA is inserted, replaced, or removed from a target DNA, e.g., the genome of a cell, using one or more nucleases and/or nickases. The nucleases create specific double-strand breaks (DSBs) at desired locations in the genome, and harness the cell's endogenous mechanisms to repair the induced break by homology-directed repair (HDR) (e.g., homologous recombination) or by nonhomologous end joining (NHEJ). The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In particular embodiments, nuclease-mediated genome editing of a target DNA sequence (e.g., a safe harbor gene) by homology-directed repair (HDR) (e.g., homologous recombination) is used for generating a genetically modified human neural stem cell in accordance with the methods described herein.

The term “DNA nuclease” refers to an enzyme capable of cleaving the phosphodiester bonds between the nucleotide subunits of DNA, and may be an endonuclease or an exonuclease. According to the invention, the DNA nuclease may be an engineered (e.g., programmable or targetable) DNA nuclease which can be used to induce genome editing of a target DNA sequence such as a safe harbor gene. Any suitable DNA nuclease can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof.

In various embodiments of the invention, a gene editing system utilizes a DNA nuclease to edit a gene and repair a mutation. In specific embodiments, a mutation is repaired using one or more of the following gene editing system: CRISPR/Cas system, Cre/Lox system, TALEN system and homologous recombination.

The differentiation protocol of the present invention may be utilized to treat a disease or disorder in a subject. The method includes administering to a subject a BFCN generated using the culturing method described herein. In various embodiments, the disease or disorder is an amyloidogenic disease, such as systemic amyloidosis, Alzheimer's disease, mature onset diabetes, Parkinson's disease, Huntington's disease, fronto-temporal dementia, and prion-related transmissible spongiform encephalopathies. In embodiments, a BFCN having a mutation that impairs neuronal excitability, such PSEN2^{N141I}, may be obtained from a subject and used to generate an iPSC, which in turn may be treated with a gene editing system to repair the mutation. The gene edited iPSC is then cultured as described herein to produce BFCNs having restored neuronal excitability which are administered to the subject to treat the disease or disorder.

In a related manner, neuronal excitability in BFCNs in a subject may be restored. This method includes: a) isolating a BFCN from the subject, wherein the BFCN has a genetic mutation resulting in impaired neuronal excitability of the BFCN; b) generating an iPSC using the BFCN of (a); c) repairing the mutation in the iPSC; d) culturing the iPSC of (c) using the differentiation protocol described herein to generate a BFCN having the repaired mutation; and e) administering the iPSC of (d) to the subject, thereby restoring neuronal excitability in BFCNs in the subject. In embodiments, the mutations is of PSEN1 or PSEN2, such as PSEN2^{N141I}.

The invention also encompasses BFCNs generated using the differentiation protocol as described herein. The BFCN may include a gene edited repair of PSEN2, as well as a detectable marker recombinantly introduced into the BFCN genome. In some embodiments, BFCNs are differentiated from PSCs, and in such embodiments, the PSCs can be iPSCs. The iPSCs can be derived from a somatic cell of a subject. In one aspect, the subject has an amyloidogenic disease or disorder.

Alongside its potential for autologous cell transplantation, iPSC technology is emerging as a tool for developing new drugs and gaining insight into disease pathogenesis. Han, S. S. W. et al., Neuron. 70:626-644 (2011). The methods and cells of the invention can aid the development of high-throughput in vitro screens for compounds that promote restoration of neuronal excitability. To that end, the disclosure provides a method of identifying a compound that can be used for treatment or prevention of a disease or disorder associated with diminished neuronal excitability in BFCNs. The method includes: a) contacting a BFCN or neuronal embryoid body (NEB) generated using the differentiation protocol described herein with a candidate compound, wherein the BFCN comprises a mutation in PSEN2 resulting in impaired neuronal excitability of the BFCN; and b) detecting neuronal excitability of the BFCN after contact with the candidate compound. A beneficial effect on neuronal excitability is evident in partial or complete restoration of neuronal excitability, thereby being indicative of a candidate therapeutic agent for treating a disease or disorder associated with diminished neuronal excitability in BFCNs, such as an amyloidogenic disease or disorder. Preferably, the method is conducted in a high-throughput format.

The invention also provides a model system for a neurological disease, preferably a disease or disorder associated with diminished neuronal excitability in BFCNs, such as an amyloidogenic disease or disorder. In one aspect, the model system comprises a BFCN differentiated from an iPSC derived from a subject having a disease or disorder associated with diminished neuronal excitability in BFCNs, such as an amyloidogenic disease or disorder. The model system can further comprise a non-human mammal into which the myelin-producing cell has been transplanted. In one embodiment, the non-human mammal is a mouse or a rat. Model systems provided by the invention can be used to study diseases or disorders, including understanding underlying mechanisms and defining therapeutic targets.

In some embodiments the present invention provides tissue culture media, tissue culture media supplements, and various kits useful in performing the various methods described herein.

In one embodiment, the invention provides a kit for generating a BFCN via the differentiation protocol of the invention. The kit includes a culture media having an inhibitor of TGF-β signaling and an activator of Shh signaling. In embodiments, the culture media is a modified mTeSR1 formulation that lacks factors that support pluripotency including bFGF, TGF-β, lithium chloride (Li—Cl), GABA and pipecolic acid. In embodiments, the culture media includes dual SMAD inhibitors, such as SB431542 and LDN193189, along with one or more agonists of smoothened protein, such as smoothened agonist (SAG) and purmorphamine.

The kit may also include an additional neuronal basal medium, such as Brainphys™ optionally supplemented with one or more of B27 supplement, an inhibitor of ROCK, NGF and BDNF.

The kit may also include reagents for detection and isolation of CD271+ cells via FACS as well as detection of expression of Tuj1, MAP2, BF1, Nkx2.1 and p75.

The kit may optionally comprise instructions for use, one or more containers, one or more antibodies, or any combination thereof. A label typically accompanies the kit, and includes any writing or recorded material, which may be electronic or computer readable form (e.g., disk, optical disc, memory chip, or tape) providing instructions or other information for use of the kit contents.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example I

### CRISPR/Cas9-Correctable Mutation-Related Molecular and Physiological Phenotypes in iPSC-Derived Alzheimer's PSEN2^{N141I} Mutation

Basal forebrain cholinergic neurons (BFCNs) are believed to be one of the first cell types to be affected in all forms of AD, and their dysfunction is clinically correlated with impaired short-term memory formation and retrieval. We present an optimized in vitro protocol to generate human BFCNs from iPSCs, using cell lines from presenilin 2 (PSEN2) mutation carriers and controls. As expected, cell lines harboring the PSEN2^{N141I} mutation displayed an increase in the Aβ42/40 in iPSC-derived BFCNs. Neurons derived from PSEN2^{N141I} lines generated fewer maximum number of spikes in response to a square depolarizing current injection. The height of the first action potential at rheobase current injection was also significantly decreased in PSEN2^{N141I} BFCNs. CRISPR/Cas9 correction of the PSEN2 point mutation abolished the electrophysiological deficit, restoring both the maximal number of spikes and spike height to the levels recorded in controls. Increased Aβ42/40 was also normalized following CRISPR/Cas-mediated correction of the PSEN2^{N141I} mutation. The genome editing data confirms the robust consistency of mutation-related changes in Aβ42/40 ratio while also showing a PSEN2-mutation-related alteration in electrophysiology.

The “amyloid hypothesis” is one of the most popular formulations for the pathogenesis of Alzheimer's disease (AD). Recent examples of clinicopathological and/or clini-coradiological dissociation have led to the consideration of alternative models in order to explain, respectively, why neuropathological AD is not always associated with dementia [24], and why about one-third of patients with clinical AD have negative amyloid brain scans [40]. It has been proposed that clinical AD can be caused by one of several “feed-forward” scenarios linking amyloidosis, tauopathy, neuroinflammation, and neurodegeneration [22]. Mutations in the gene encoding presenilin 2 (PSEN2) are associated with autosomal dominant early onset familial Alzheimer's disease (EOFAD). The linkage of a locus on human chromosome 1q31-42 linked to EOFAD led to the identification of the PSEN2^{N141I} point mutation in the Volga German kindreds in 1995 [43]. This mutation causes elevation in the Aβ42-43/40 ratio, thereby promoting assembly of Aβ oligomers and fibrils [83].

In considering the progression of AD, human basal forebrain cholinergic neurons (BFCNs) are one of the first cell types whose dysfunction underlies the early loss of short-term memory recall in all forms of AD. The “cholinergic hypothesis of AD” was formulated in the mid-1970s [6, 20, 61], and the discoveries of reduced acetylcholine release from neurons of the nucleus basalis of Meynert confirmed the presence of a presynaptic cholinergic deficit in the basal forebrain of AD patients [1, 71]. Based on those observations, acetylcholinesterase inhibitors were developed and continue as the most widely used symptomatic treatments for AD [21, 28, 33, 82]. Eventually, post-mortem brain biochemical and volumetric studies at different stages of the disease identified several other regions of the brain that were also affected early in the course of AD [63]. These studies have traditionally focused on the hippocampus and cortex, but more recently, attention has begun shifting back to the basal forebrain and adding other areas, such as the striatum [27, 62]. The latest analyses suggest that cholinergic basal forebrain volume measurement may be a better predictor of the transition from MCI to AD than the previous standard, hippocampal volume [10].

We previously reported the generation of iPSC-derived neurons from banked fibroblasts from subjects harboring PSEN1^{A246E} and PSEN1^{M146L} mutations [77]. In characterizing the gene expression profiles from these iPSC-derived neurons, we observed an unexpected association of elevated expression of the inflammasome gene NLRP2 in undifferentiated PSEN1 mutant iPSCs and their and neuronally differentiated progeny [77]. This led us to examine NLRP2 expression in our PSEN2 mutant lines and employ CRISPR/Cas9 [15] to investigate if activation of the inflammasome was tightly linked to the pathogenic mutation in PSEN2. While we did not find altered expression of NLRP2 in gene-corrected PSEN2 lines, we observed significant mutation-related, editing-reversible differences in excitability of BFCNs.

### Materials and Methods

Generation and Maintenance of iPSC Lines

7889(s)B, 050643 (Control), 948 (AD1), 949(fControl), and 950 (AD2) iPSC lines were obtained via the NYSCF Repository following the guidelines from [60]. The derivation and characterization of Nkx2.1-GFP ESC line was previously published [30]. ES and iPS cell lines were expanded and maintained in serum-free mTeSR1™ media (Stem Cell Technologies). Cells were lifted using StremPro™ Accutase (ThermoFisher) and media was supplemented with 10 μM ROCK inhibitor (Y27632, Stemgent) during cell passaging.

For all studies in this paper, cell lines underwent at least 3 independent differentiations from the iPSC stage to the mature neuron stage. Data were routinely compared across these independently derived genotype-identical neurons (or in some cases neuronal precursors), and if comparable results were obtained across independently genotype-identical derived cells, they were considered to be qualified representatives of their genotype and so were passed along for genotype-specific experimentation.

### Aβ42 Oligomer Preparation

Aβ42 oligomers were prepared as previously reported [23, 78]. Briefly, we dissolved 1 mg of Aβ42 (American Peptide Company) in 1,1,1,3,3,3-hexafluoro-2-propanol (HFIP) (Sigma). This preparation was aliquoted and dried using a SpeedVac™ centrifuge. The pellet was then resuspended in DMSO to obtain a 5 mM solution which was sonicated in a water bath for 10 min. From here aliquots were stored at −20 C and used within 2 weeks by diluting with 100 μl of PBS and leaving for 12 h at 4° C. in order for oligomerization to proceed. This final solution was diluted 1:16 in cell media for studies, allowing cells to be exposed to 5 μM of Aβ42 oligomers. Control wells were diluted with 1:16 PBS. Cells were exposed to oligomers or PBS without media change for a period of 3 days.

### Cell Death Assays

Cells were assayed in a 96-well plate format. Oligomer or vehicle solutions were added to media and allowed to incubate for a period of 3 days. Media was then collected and assayed using a lactate dehydrogenase toxicity assay (Thermo Fisher Scientific). 50 μl of media and an equal amount of reaction mix buffer were incubated for a period of 30 min. An additional set of wells per experiment were treated with 2% Triton™ X-100 for a 5-min period in order to lyse all cells, and media from these wells was also collected and incubated as described. After incubation absorbance was recorded at 490 nm and 680 nm, signal and background absorbance, respectively. Signal values were subtracted from background, and values were adapted to the total LDH content as determined by Triton X-100 treated wells. Propidium iodide (Thermo Fisher Scientific) was added to cell media for a 1 μM final concentration and allowed to incubate for 5 min. Cells were then washed twice with media and imaged. Images were captured using CELIGO™ image cytometer and accompanying software (Nexcelom Bioscience). Each biological variable was assessed in technical triplicates within each designated “Experiment”, and each designated “Experiment” was performed in at least three complete “start to finish” iterations.

Differentiation of Basal Forebrain Cholinergic Neurons from iPS and ES Cells

Human ES or iPSC were plated as single cells after chemical dissociation using Accutase™ (Sigma-Aldrich) into Cul-Trex™ (Trevigen) coated plates, at a density of 4-8×10⁵ cells per well in 6-well plates or petri dishes and adapting cell numbers. Cells were initially maintained in mTeSR1™ media (Stem Cell Technologies) until reaching full confluency. On “day 0” of differentiation, media was replaced by Custom mTeSR1™ media (Stem Cell Technologies) lacking factors promoting pluripotency i.e., bFGF, TGF-β, Li—Cl, GABA and pipecolic acid. The addition of dual SMAD inhibitors (SB431542 10 μM plus LDN193189 250 nM, Selleckchem) at day 0 drives cells towards neuroectoderm specification. At day 2 of differentiation, media was replaced by Custom mTeSR1 containing dual SMAD inhibitors plus two ventralizing agents: SAG at 500 nM (R&D) and Purmorphamine at 2 μM (Stemgent™). Cells were fed every 2 days with this media until day 9, when media was progressively switched to Brainphys™ media (Stemcell Technologies) supplemented with B27 (Life Technologies) [3]. Neural progenitors were harvested at day 11 using Accutase, p75+(CD271) NPCs were purified by FACS and plated at a density of 80,000 cells per well into non-adherent 96 well V-bottom plates in Brainphys™+B27 supplemented with 10 μM ROCK inhibitor (Y27632, Stemgent), Nerve Growth Factor, NGF (Alamone labs, 50 ng/mL) and Brain Derived Neurotrophic Factor, BDNF (R&D, 50 ng/mL). Cells were allowed to aggregate and form Neuronal Embryoid Bodies (NEBs) and were fed every other day until day 19. At day 19 NEBs were dissociated using Accutase (Sigma-Aldrich) and were plated as monolayer cultures on plates coated with branched polyethynilimine (0.1%, Sigma-Aldrich) and laminin (10 mg/mL, Life Technology) in Brainphys™ media+B27 supplement with BDNF and NGF. The media was changed every 2 days until analysis. As an alternative, 3D NEBs were dissected manually into 3-4 pieces for expansion and further grown, or were cryopreserved. Initial versions of the protocol used Neurobasal™ as a base media instead of Brainphys™.

### Genomic DNA Isolation and Sequencing

Genomic DNA was isolated from PSEN2 mutant or control iPSC lines using High Pure™ PCR Template Preparation Kit (Roche) following manufacturer instructions. Genomic samples were treated with RNAse (QIAGEN) prior to amplification. A fragment from exon 5 of PSEN2 containing PSEN2^{N141I} mutation was amplified using the following primers: Forward 5′-CATCAGCCCTTTGCCTTCT-3′ (SEQ ID NO: 3), Reverse: 5′-CTCACCTTGTAGCAGCGGTA-3′ (SEQ ID NO: 4), generating a 173 bp fragment, regardless of the genotype. For detection of ApoE allelic variants, a fragment of 244 bp was amplified prior to sequencing using the primers: Forward: 5′-ACAGAATTCGCCCCGGCCTGGTACAC-3′ (SEQ ID NO: 5), Reverse: 5′-TAAGCTTGGCACGGCTGTCCAAGGA-3′ (SEQ ID NO: 6). Both PCR were performed with the following settings: 10 min 94 C, 40 cycles (30 s 94 C, 20 s 62 C, 10 s 72 C) 7 min 72 C. PCR products were run in a 2% agarose gel to check the size of the amplified fragment. After amplification, samples were cleaned using EXOSAP-It™ (Thermo Fisher Scientific) and then sequenced using the following primers: PSEN2 (Forward: 5′-TCAGCATCTACACGCCATTC-3′ (SEQ ID NO: 7), Reverse: 5′-AGCACCACCAAGAAGATGGT-3′) (SEQ ID NO: 8), from [53]; ApoE (Forward: 5′-ATTCGCCCCGGCCTGGTACAC TGCCA-3′ (SEQ ID NO: 9), Reverse: 5′-CTGTCCAAGGAGCTGC AGGCGGCGCAG-3′ (SEQ ID NO: 10)), from [36].

### CRISPR/Cas9 Gene Correction

iAD1 Control and iAD2 Control lines were originated from 948 (AD1) and 950 (AD2) iPSC lines by CRISPR/Cas9-mediated correction of the PSEN2^{N141I/WT} heterozygous point mutation to PSEN2^{WT/WT}. g1N141I single guide RNA (sgRNA) was cloned into pSpCas9(BB)-2A-GFP (PX458) vector, generating pSpCas9-g1N141I-GFP vector to direct gene editing to the sequence in exon 5 of PSEN2 where the Volga mutation is located. Single stranded oligonucleotides (ssODN) are efficient templates for the CRISPR/Cas9 correction [13, 66]. ssODN #A-N141I (sequence detail below) was used as donor sequence for gene correction. We designed asymmetric ssODN sequences with a long homology arm of 91 bp, and a short homology arm of 36 bp since asymmetrical ssODNs showed a higher efficiency of homology-directed repair using CRISPR/Cas9

TABLE 1

| Sequence | | | SEQ |
|---|---|---|---|
| Name | Bases | Sequence | ID NO |
| g1N141I | 25 | /5Phos/CACCGCATCATGATC | 11 |
| guide RNA F | | AGCGTCATCG | |
| g1N141I | 25 | /5Phos/AAACCGATGACGCTG | 12 |
| guide RNA R | | ATCATGATGC | |
| Donor | 127 | GAGAGAAGCGTGGCTGGAGGGC | 13 |
| ssODN#A | | AGGGCCAGGGCCTCACCTTGTA | |
| N141I | | GCAGCGGTACTTGTAGAGCACC | |
| | | ACCAAGAAGATGGTCATAACCA | |
| | | CGATGACGCTGATCATGATGAG | |
| | | GGTGTTCAGCACGGAGT | |

Underline = Base of the ssODN that Corrects the Point Mutation

The donor sequence and pSpCas9-g1N141I-GFP vector were transduced in the AD1 and AD2 iPSC lines, plated at 50-70% confluency, using Amaxa Human Stem Cell Nucleofector™ kit (Lonza VPH-5002) and re-plated for recovery. GFP⁺ cells were sorted in a BD FACSAria IIu Cell Sorter™ and were seeded at 30-50 cells per well in 96-well format to detect and pick single clones. Positive clones were expanded, qDNA was extracted and analyzed for successful HDR was determined using a custom designed TaqMan™ genotyping assay with a probe specific for the SNP (dbSNP ID: rs63750215) located in Chr1:227,073,304 A>T. Selected clones were analyzed by Sanger sequencing to confirm correction of Chr1:227,073,304 location and discard possible insertions or deletions in the surrounding areas.

### Fluorescence-Activated Cell Sorting (FACS)

Neural progenitors at day 12 of differentiation were dissociated with Accutase (Sigma-Aldrich) for 5 min at 37 C and inactivated in Neurobasal media. Cells were spun at 1000 rpm for 4 min and the pellets were resuspended in FACS buffer (DPBS, 0.5% BSA Fraction V-Solution, 100 U/mL Penicillin-Streptomycin, 0.5%. EDTA and 20 mM Glucose) with PE Mouse anti-human CD271 antibody (clone C40-1457, BD) at 1:100, also known as p75 or NGFR, and incubated for 20 min at room temperature (RT) in the dark. After the incubation time, cells were washed with FACS buffer and the pellet was resuspended in 2 mL of FACS buffer with 10 μM ROCK inhibitor (Y27632, Stemgent). p75 positive cells were purified in a BD FACSAria IIu Cell Sorter™ and data was analyzed using FlowJo™ software.

### Real-Time Quantitative Polymerase Chain Reaction (RT-qPCR)

Human iPSC from PSEN2 mutants or control patients were grown in a monolayer and lysed directly in the cell culture wells with RLT buffer. Total RNA purification was performed with the RNeasy™ Micro kit (Qiagen), and was carried out according to the manufacturer's instructions. cDNA was synthesized using SuperScript™ III Reverse Transcriptase (RT) (Invitrogen, Carlsbad, CA). Semi-quantitative real-time PCR was performed on StepOnePlus™ Real-Time PCR System (Applied Biosystems, Foster City, CA) using the primers listed in Table 2 below. We normalized expression levels to GAPDH. The PCR cycling parameters were: 50° C. for 2 min, 95° C. for 10 min, followed by 40 cycles of 95° C. for 15 s and 60° C. for 1 min. Each biological variable was assessed in technical triplicates within each designated “Experiment”, and each designated “Experiment” was performed in at least three complete “start to finish” iterations. Expression levels were normalized to the control line, and results were expressed as AVG±SEM.

TABLE 2

| | Forward | SEQ | Reverse | SEQ |
|---|---|---|---|---|
| | Primer | ID | Primer | ID |
| Gene | 5′-3′ | NO | 5′-3′ | NO |
| BDNF | TAACGGCGGCAGAC | 14 | GAAGTATTGCTTCAG | 15 |
| | AAAAAGA | | TTGGCCT | |
| BF1 | AGAAGAACGGCAAG | 16 | TGTTGAGGGACAGAT | 17 |
| | TACGAGA | | TGTGGC | |
| Nkx2.1 | TAACGGCGGCAGAC | 18 | GAAGTATTGCTTCAG | 19 |
| | AAAAAGA | | TTGGCCT | |
| NLRP2, | From [77] | 20 | From [77] | 21 |
| ASB9 | | | | |
| NLRP3 | ACGAATCTCCGACC | 22 | CCATGGCCACAACAA | 23 |
| | ACCACT | | CTGAC | |
| Tuj1 | GAAGTGTCCCAGGA | 24 | CTCTTGAGTAGCTGG | 25 |
| | CATGATAA | | GATTGAG | |

### Aβ Assays

Cells were conditioned for 3 days after day 8 of dual SMAD inhibition to measure secretion of A3 by neural progenitors in vitro. A3 levels were quantified using human/rat 3 amyloid 40 ELISA Kit and 3 amyloid 42 ELISA Kit high sensitive (Wako). Each biological variable was assessed using technical triplicates within each designated “Experiment”, and each designated “Experiment” was performed in at least three complete “start to finish” iterations.

### Immunostaining/ICC

Cells were fixed with PFA 4% directly on the wells of 12, 48 or 96 well plates for 20 min, washed 3 times with DPBS 1×(ThermoFisher). For the staining, cells were incubated in blocking solution (DPBS 1× with 0.1% Triton™ X-100 plus 5% Donkey serum) for two hours at room temperature. The corresponding primary antibodies were diluted at suitable concentration in blocking solution, and incubated overnight at 4 C. The primary antibodies used are represented in the table below. Cells were washed three times with DPBST (DPBS 1×+0.1% Triton™ X-100) and suitable secondary antibody was added in blocking solution for 1 h at room temperature. Then cells were washed three times with DPBST and incubated with DRAQ5 or Hoescht 33,342 (1 μg/mL, diluted in DPBS 1×) for 10 min at room temperature for nuclear counterstain. Cells were visualized using an inverted fluorescence microscope (Olympus™ IX71 microscope) or a confocal microscope (Zeiss™ LSM5 Pascal microscope) under 10×, 20× or 63× magnification.

### Western Blots

Human iPSC from PSEN2 mutants or control patients were grown in a monolayer and lysed directly in the cell culture wells with RIPA buffer (Thermo Scientific) with protease and phosphatase inhibitors. The protein concentration was measured using the BCA protein assay kit (Thermo Scientific). After protein estimation, 20 μg of cell lysate were separated by SDS-PAGE electrophoresis on a 4-12% Bis-Tris gel (Bolt™ protein gels) and transferred onto nitrocellulose membranes by electrophoresis blotting. The membranes were blocked with blocking buffer 1×TBST (tris-buffered saline+0.10% Tween) plus 5% nonfat dry milk for 1 h in agitation at room temperature and washed three times with TBST. After washing, membranes were incubated at 4° C. overnight in agitation, with the primary antibodies against NLRP2 (1:1000), PSEN2 (1:200) or 3-actin (1:1000). After rinsing, the membranes were incubated with horseradish peroxidase (HRP)-conjugated suitable secondary antibodies for 1 h at room temperature. Finally, protein bands were visualized with a chemiluminescent reagent according to the manufacturer's instructions. 3-actin was used as loading control. Electrophysiology

Whole cell patch-clamp recordings were obtained from single neurons between differentiation days 38 and 55. Cells were seeded at low density onto plastic coverslips which were placed in a perfusion based enclosed recording chamber. Neurons were localized using differential interference contrast optics under an Olympus BX61WI microscope fitted with a Hamamatsu Orca™ R² CCD camera. Recordings were carried out at room temperature using MultiClamp™ 700B amplifier (Molecular Devices, Sunnyvale, CA, USA). Signals were sampled at 10 kHz and filtered at 6 kHz using a Digidata™ 1440A analog to digital converter (Molecular Devices). Amplifier control and data acquisition was done using pClamp™ 10.0 software (Molecular Devices).

During recordings neurons were perfused with oxygenated Brainphys' media (StemCell Technologies Inc). Medium resistance recording pipettes (4-6 MΩ) were filled with an intracellular solution consisting of (in mM) 130 K-gluconate, 10 KCl, 2 Mg-ATP, 0.2 Na-GTP, 0.6 CaCl2), 2 MgCl2, 0.6 EGTA, and 5 HEPES titrated to pH 7.1 and osmolarity of 310 mOsm. In some experiments, the intracellular solution also contained 4 mg/mL biocytin (Sigma-Aldrich) for post-hoc identification of individual neurons, which were visualized with streptavidin-conjugated Alexa 488 (Life Sciences) as described elsewhere [42]. After initial break-in, access resistance (Rs) was constantly monitored and recordings were discarded if Rs exceeded 20 MΩ or changed more than 30%. The voltage protocol for compound Na+ and K+ currents characterization was as follows: cells were held at −80 mV potential followed by 500 ms steps from −100 mV to 30 mV with 10 mV increment at a frequency of 0.1 Hz. Following transition to current-clamp mode, resting membrane potential was recorded and cells were hyperpolarized by a negative DC current injection to −70 mV to ensure consistency of excitability measurements. Action potentials were evoked with square 1 s current steps from −10 pA to 40 pA with 1 pA steps.

Electrophysiological recordings were analyzed using ClampFit™ software (Molecular Devices, Sunnyvale, CA, USA) and statistical significance of the results was measured using ANOVA test with Tukey's post-hoc comparison of means. Salts and other reagents were purchased from Sigma-Aldrich (St. Louis, MO, USA).

### Statistical Analysis

qPCR gene expression experiments and Aβ42/40 ELISAs were analyzed for statistical significance using Student t-test. LDH Release assays were analyzed by 2-Way ANOVA Bonferroni post hoc tests. ANOVA test with Tukey's post hoc comparisons were used for analysis of electrophysiology results. The experiments needed to study each of the 94 neurons recorded for electrophysiology analyses required days to weeks. On each experimental day, representatives from each genotype were included, with at least three samples from each genotype studied on each day. *, p<0.05; **, p<0.01; ***, p<0.001.

### Results

### Optimization of Protocol for BFCN Differentiation

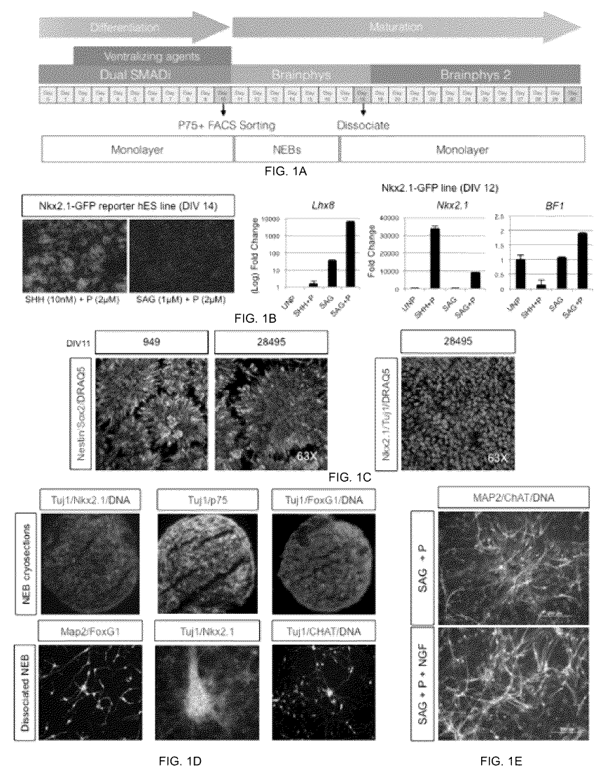

The scheme of BFCN differentiation is described in FIG. 1A. iPSCs from control subjects or AD patients were plated in feeder-free conditions and allowed to reach 100% con-fluency prior to differentiation using mTeSR1 basal media. Both branches of TGF-β signaling were inhibited (dual SMAD inhibition) to induce neuroectodermal fate on “day 0” [12]. Differentiations (day 2-10) were performed using a modified mTeSR1 formulation, lacking factors that support pluripotency (bFGF, TGF-β, Li—Cl, GABA and pipecolic acid). To specify these cells to basal forebrain cholinergic neurons, ventralization for medial ganglionic eminences (MGE) induction is required [19, 85, 91]. As such cells were treated with the Sonic Hedgehog (Shh) analog (SAG) at 500 nM and Purmorphamine at 2 μM from days 2 to 8. SAG is a suitable substitute to activate Shh signaling, as demonstrated during differentiation of ChAT⁺ motor neurons and glutamatergic interneurons [91], with lower cost than recombinant Shh and some advantages in neuronal survival properties over Shh itself [7, 35]. We used the Nkx2.1-GFP embryonic stem cell (ESC) reporter line as a tool to adjust the combination, dosage and timing of ventralizing agents more beneficial for specification of BFCNs from induced Nkx2.1 basal forebrain precursors.

However, given the potential of Nkx2.1 intermediate neural precursors to generate multiple neuronal subtypes, such as TH+ and GABA+ hypothalamic neurons, we analyzed the expression of the downstream cholinergic specification factor Lhx8 over expression of the GABAergic interneuron specific transcription factor Lhx6 [26] under different specification conditions (FIG. 1B). These data agree with those from [50] supporting the existence of a synergistic effect of SAG and purmorphamine on Nkx2.1 induction although an effect that is less than the effect of Shh plus purmorphamine (FIG. 1B). Nkx2.1-driven GFP levels were maintained after Day 14, even after withdrawal of SAG+purmorphamine at day 8 (FIG. 1B). We observed higher Lhx8 induction upon SAG plus purmorphamine treatment than SAG alone, or even Shh plus purmorphamine (FIG. 1B). Interestingly, intermediate Nkx2.1 levels driven by SAG plus purmorphamine correlate with higher induction of Lhx8 and BFT gene expression (FIG. 1B). Our choice of starting SHH pathway-driven ventralization at day 2 was based on reports demonstrating other MGE-derived populations being generated by earlier (e.g., hypothalamic neurons) or later (e.g., GABAergic interneurons.) ventralization in the context of dual smad inhibition protocols.

Following the patterning stage, we gradually switched from Custom mTESR1™ media to Brainphys™ media with B-27 supplement to support neuronal survival and growth [3]. At day 11, we observed neural rosettes positive for Nestin and Sox2 markers (FIG. 1C); also, we observed Tuj1+ neurites as early as day 11 (FIG. 1C). To obtain cholinergic populations of a higher purity, we developed a P75+ FACS strategy to isolate progenitors specific for cholinergic neurons due to the fact that BFCNs are the only CNS neuron type to express robust levels of P75 under non-pathogenic conditions in the adult brain). Support for this strategy includes a previously published protocol using FACS to isolate high expressing P75+ cells from the embryonic murine septum [65]. This population correlated with best expression of cholinergic-related markers.

At day 11/12, we lifted the cells using chemical dissociation (Accutase) and purified day 11-12 p75+(CD271) neural progenitors and generated 3D neuronal embryoid bodies (NEBs) by spinning down neural progenitors in V-bottom 96 well plates. On day 19 NEBs were dissociated and re-plated as a monolayer on plates coated with branched polyethylenimine (Aldrich catalog number 408727) and laminin. Monolayer cultures were maintained with the addition of growth factors BDNF, NGF and DAPT until day 26, when cultures no longer had DAPT added. Immunostaining of both cryosections of NEB structures and fixed monolayers, resulting from chemical dissociation of NEBs from several control iPSC and H9 hESC lines, demonstrated expression of BFCN lineage markers Tuj1, MAP2, BF1, Nkx2.1 and p75, at final stages of the differentiation protocol (FIG. 1D). NGF addition to neuronal cultures showed an advantageous effect on maturation, neurite outgrowth and presence of ChAT (FIG. 1E).

Generation and QC of PSEN2^{N141I} iPSC Lines

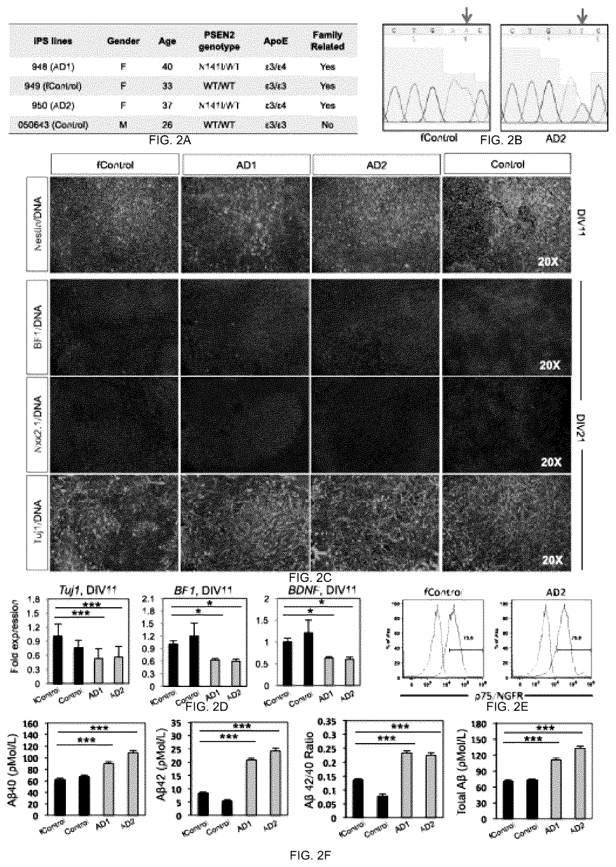

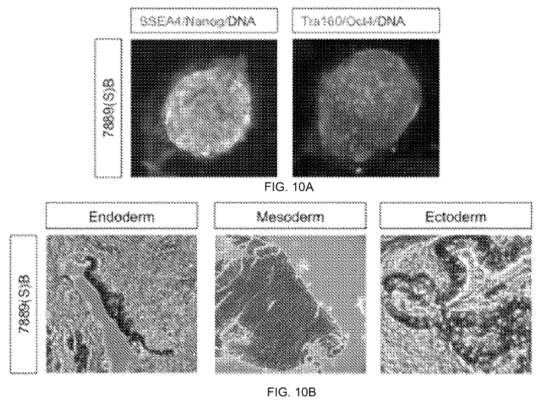

PSEN2^{N141I}, mutant iPSC and control lines were generated from fresh skin biopsies. Established fibroblast lines were grown from skin punches donated by a kindred of 2 carriers for presenilin 2 Volga familial AD mutation (PSEN2^{N141I}) and one non-affected member. Additionally, we included a non-family related control. Fibroblasts were reprogrammed using modified RNA method to introduce the Yamanaka factors (Oct4, KLF4, SOX2 and c-Myc), and the iPSC lines obtained were subject to several quality control processes to ensure robust cell-renewal and pluripotency, including alkaline-phosphatase (AP) enzymatic activity, gene expression analysis and immunostaining for pluripotency markers, as well as karyotyping for detection of chromosome abnormalities, following the automated iPSC reprogramming and QC methods developed by [60]. A summary of the genotypes, sex and age of the subjects included in the study is shown in FIG. 2A. The two PSEN2^{N141I}, iPSC lines were also heterozygous for APOE ε4 (ε3/ε4), whereas the control iPSC lines were homozygous ε3/ε3. The characterization of the iPSC lines, expression of pluripotency markers and quality control results are shown in FIGS. 10A-10B. Briefly, all iPSC clones selected demonstrated pluripotency by embryoid body formation and differentiation into the three germ layers (FIG. 10A), incorporated herein by reference). Finally, the lines were fingerprinted (Cell Line Genetics) to ensure they matched the parental fibroblast lines (data not shown). All the parental fibroblast lines and the iPSC lines were subject to Sanger sequencing to determine PSEN2 and APOE genotypes. A 173 bp fragment from the exon 5 of PSEN2, surrounding the area where the PSEN2^{N141I} point mutation is located (Chr1:227,073,304 A>T), was amplified by PCR and sequenced using the primers published in [53]; similarly a fragment of 244 bp from APOE locus that contains two SNPs which determine the three allelic variants was amplified by PCR from genomic DNA, and subsequently sequenced to distinguish between 82/83/84 variants, using the primers from [36]. Sample chromatograms showing the presence of PSEN2N141I point mutation are shown in FIG. 2B, and all genotypes are summarized in FIG. 2A.

### Characterization of PSEN2^{N141I} Neural Progenitors

To study the effect of the PSEN2^{N141I} mutation in early stages of the differentiation of cholinergic neurons, we analyzed the neural progenitors (NPCs) obtained at DIV 11-16 along the BFCN differentiation protocol. The analysis of this intermediate immature population allows us to detect possible early alterations in the generation of BFCNs that would otherwise not be detected in terminally differentiated cholinergic neurons. Such defects could potentially play roles in mature neurons and contribute to the pathophysiology of AD. We analyzed the expression of early neuronal markers in PSEN2^{N141I} mutant and control NPCs by gene expression and immunofluorescence methods. Although, we found a lower RNA expression of Tuj1 (βIII-Tubulin), a general neuronal marker, in mutant NPCs at day 11 of differentiation, we did not detected quantifiable differences by immunocytochemistry circa days 16-21 (FIGS. 2C and 2D). NPC monolayer cultures at day 11 were also immunostained for typical NPC markers: Sox2, and Pax6; with Pax6 levels dropping as expected along with Nkx2.1 induction (not shown). We observed comparable expression of Sox2 and Nestin in PSEN2^{N141I}, cultures at day 11 (FIG. 2C, top panel). At day 21, mutant NPCs expressed comparable levels of Nkx2.1 (MGE marker), but reduced levels of BF1 (forebrain marker) by qPCR; however, BF1 protein expression did not seem affected by immunostaining at this differentiation stage (FIG. 2C bottom panel, and D). We did not observe differences in the surface expression of NGFR (p75/CD271) in DIV11-12 PSEN2^{N141I} cells, in terms of percentage of positive cells or fluorescence mean peak value (FIG. 2E).

As previously published by [59, 73], the expression of mutant PSEN2^{N141I} causes an increase in the Aβ42/40 ratio in the brains of transgenic mice; additionally, this enhanced Aβ42 production was observed in neural cell lines upon induced overexpression of mutant PSEN2^{N141I} protein [83] and in iPSC derived from PSEN2^{N141I} mutant patients [93]. Consistently, we observed a 2-fold increase in the Aβ42/40 ratio, a 50% increase in the amount of secreted Aβ40 and 2.5-fold increase in Aβ42 species in the conditioned media from PSEN2N141′ neural progenitors at DIV 11 (***p<0.001) (FIG. 2E). The levels of secreted Aβ40 and 42 observed in our study and the levels found in [93], using a different neuronal differentiation method applied to FAD1/PS2 iPSC lines derived from fibroblasts from the Coriell repository are very similar in both absolute number and in fold-increase.

Characterization of Mature BFCNs from PSEN2^{N141I} iPSC Lines and Controls

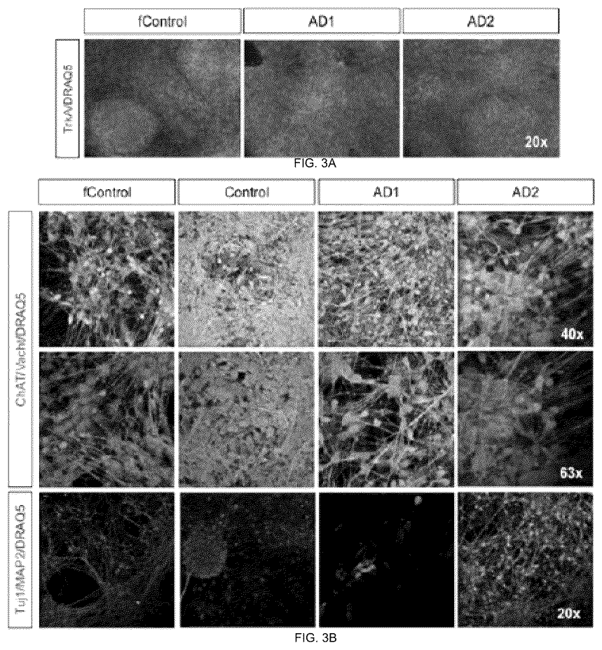

With the aim of determining the impact of PSEN2^{N141I} mutation on the differentiation, gene expression, function, and communication of BFCNs, we characterized cells at later time points for appropriate expression markers; our goal was to explore whether PSEN2^{N141I} iPSC were able to complete BFCN maturation process and if so, if any abnormalities along later stages of BFCN differentiation may account for the pathophysiology of EOFAD (FIGS. 3A-3B). In addition to p75, which preferentially binds pro-NGF, we analyzed the expression of TrkA, the primary mature NGF receptor, was also expressed in PSEN2^{N141I} BFCNs and control (FIG. 3A). This suggested that PSEN2^{N141I} BFCNs are susceptible to receiving and benefiting from NGF pro-survival and differentiation signals as expected and further confirms their proper identity. We observed comparable expression of additional cholinergic neuron specific markers choline acetyltransferase (ChAT) and vesicular acetylcholine transporter (vAChT) in PSEN2^{N141I} BFCNs and controls (FIG. 3B). Other general neuronal markers such as Tuj1, and the mature marker microtubule-associated protein 2 (MAP2) showed no apparent differences by immunofluorescence (FIG. 3B).

### CRISPR/Cas9-Mediated Correction of PSEN2^{N141I} Mutation and Effect on Aβ 42/40 Ratio

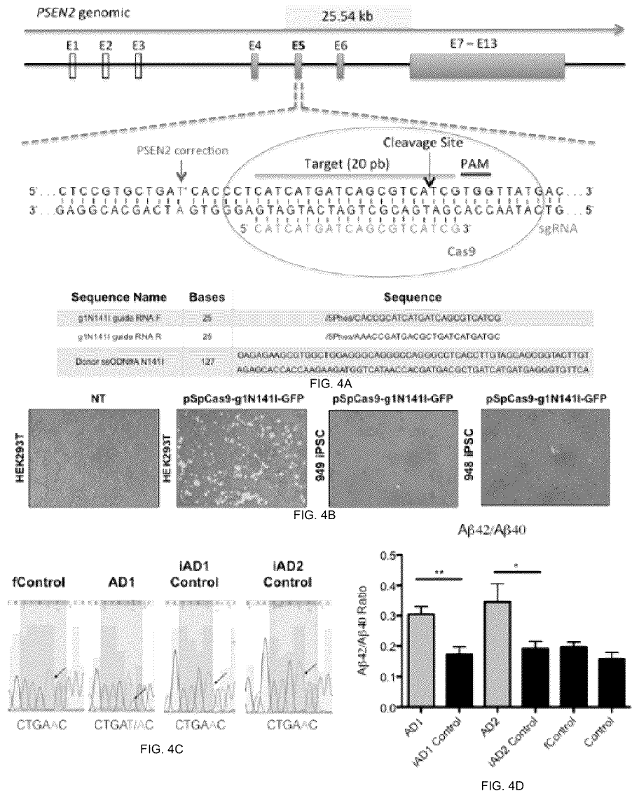

To determine if the molecular alterations in the processing and cleavage of APP and/or the exacerbated activation of NLRP2 inflammasome, as previously observed in PSEN1 mutants [77], can be attributed to PSEN2^{N141I} mutation only, we modified the PSEN2 locus in our iPSC lines employing CRISPR/Cas9 technology. We did this by correcting the PSEN2^{N141I} point mutation in the two PSEN2 mutant iPSC lines (AD1, AD2). For this purpose, a specific guide RNA (g1N141I) was designed using an online tool (tools.genome-engineering.org) to direct Cas9 to the region of PSEN2 exon 5 surrounding PSEN2^{N141I} mutation (23 bp upstream of Chr1:227,073,304 A>T). g1N141I was cloned into pSpCas9(BB)-2A-GFP (PX458) vector. Expression was assessed by GFP fluorescence upon transfection of pSpCas9-g1N141I-GFP in HEK293T (FIG. 4A).

In order to correct the mutation, we designed an asymmetric ssODN HDR (homology directed repair) template, ssODN #A-N141I, with a long homology arm of 91 bp, and a short homology arm of 36 bp since asymmetrical donor sequences with a shorter arm oriented to the area closer to the PAM side demonstrated a superior efficiency of homology-directed repair using CRISPR/Cas9 system [13]. We then proceeded to trans-duce pSpCas9-g1N141I-GFP and ssODN #A-N141I into the iPSC lines using Amaxa™ nucleofection (FIG. 4A). Forty-eight hours post-nucleofection cells were dissociated and the GFP⁺ population was purified by FACS and replated at low density feeder free for isolation of single gene-corrected clones (FIG. 4B). Subsequently, clones were grown and gDNA extracted post expansion. The screening of positive clones that demonstrated successful HDR was determined by qPCR using a custom designed TaqMan™ genotyping assay with a probe specific for the SNP (dbSNP ID: rs63750215) located in Chr1:227,073,304 A>T. We were able to distinguish by this method between homozygous PSEN2^{N141I}, heterozygous PSEN2^{N141I} and PSEN2^{WT} single clones derived from the original iPSC lines, and pre-selected clones were subjected to Sanger sequencing to confirm Chr1:227,073,304 location and detect possible insertions, deletions or mismatches introduced by CRISPR/Cas9 modification in the surrounding area and corroborate successful HDR (FIG. 4C).

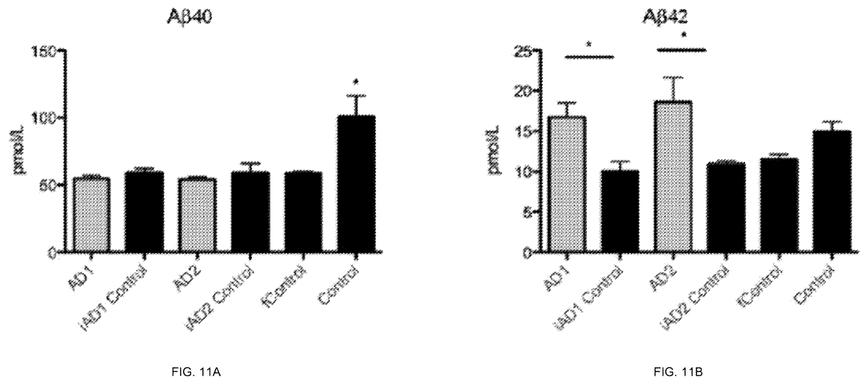

Successfully corrected clones were expanded and subjected to the BFCN differentiation protocol in parallel to the other 4 lines used in the study. We collected media from BFCNs (DIV 34) and re-tested for amyloid beta production. In support of our previous finding in NPCs at DIV 11-12 (FIG. 2F), we observed that mature BFCNs also display significant increases in Aβ42/40 ratio (FIG. 4D) and overall A3 production (FIGS. 11A-11B). Importantly, these results also showed a normalization of Aβ42/40 ratio to control levels in corrected lines (iAD1 Control and iAD2 Control, are corrected clones of AD1 and AD2, respectively) (FIG. 4D). These results also strengthen previous findings linking the PSEN2^{N141I} mutation to abnormal APP processing and reinforcing that presenilins contains the catalytic site of γ-secretase [90].

Assessment of Sensitivity to Aβ42 Oligomer Toxicity in iPSC-Derived PSEN2^{N141I} Neurons

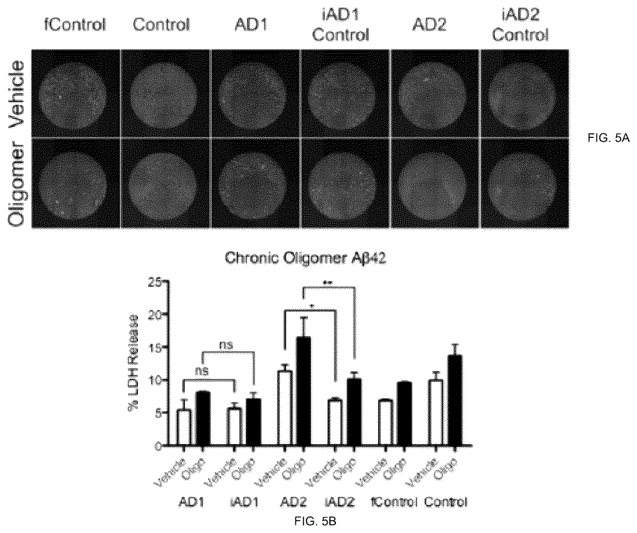

Previous reports have shown that iPSC lines carrying FAD mutations may display an enhanced susceptibility to noxious stimuli, such as high concentrations of Aβ42 oligomers [2]. We therefore tested whether our BFCNs from PSEN2^{N141I} mutants would display enhanced toxicity to Aβ42 oligomers in the media (FIGS. 5A-5B). We assessed neurotoxicity by measuring the percentage of lactate dehydrogenase (LDH) released by dead cells, thus providing an indirect measurement for toxicity. Using this methodology by 2-way ANOVA we detected a significant effect in toxicity driven by 5 μM Aβ42 oligomer addition to the culture media, after 72-h exposure (***, p<0.01). Post hoc Bonferroni analysis revealed significant differences between the AD2 line and its corrected isogenic control (iAD2 Control). However, this apparent enhanced sensitivity to Aβ42 oligomer toxicity was not observed in the AD1 line and its corresponding control. These results indicate that differences in susceptibility to Aβ42 are not exclusively linked to mutant PSEN2 genotype, and that likely additional genetic factors different between AD1 and AD2 subjects affect susceptibility to this stress, further emphasizing the importance of multiple isogenic models.

Assessment of NLRP2 mRNA in iPSC-Derived PSEN2^{N141I} Neurons

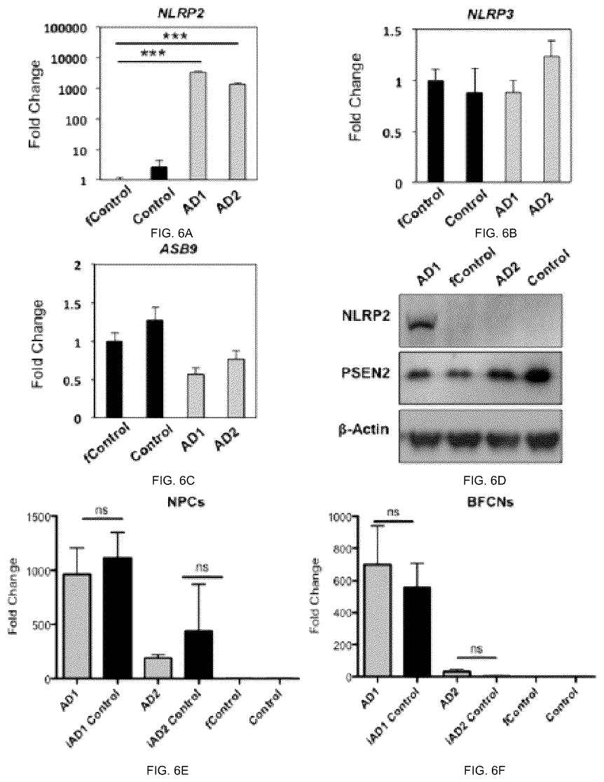

We previously reported that NLRP2 mRNA was elevated in PSEN1 mutant iPSC and NPCs, [77] which was also the case for PSEN1 mutant cortical neurons (unpublished observation). Therefore, we wanted to analyze the status of the components of the inflammasome in the context of PSEN2^{N141I} mutation. When we assayed by qPCR the mRNA levels of NLRP2 in NPCs at DIV12, we observed an increase over 100-fold in AD1 and AD2 lines, as compared to control lines (FIG. 6A). This correlated with a notable increase in NLRP2 protein, as observed by SDS-PAGE in whole cell lysates from day 11 PSEN2 mutants (FIG. 6D). Noticeably, however we did not detect bands for NLRP2 by immu-noblot in the AD2 line lysates. Further, we were unable to corroborate some other transcriptional events previously seen in PSEN1 mutant iPS neural precursors, such as the elevated ASB9 that encodes an E3 ligase that directs mitochondrial creatine kinase for degradation. Instead, we observed a trend toward decreased levels in PSEN2 mutation carriers by 20-30%.

Assessment of Excitability of iPSC-Derived PSEN2N141I BFCNs

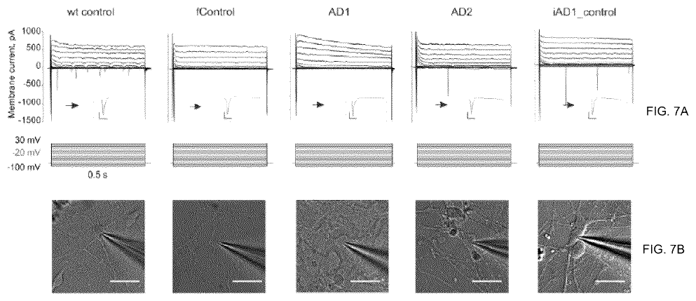

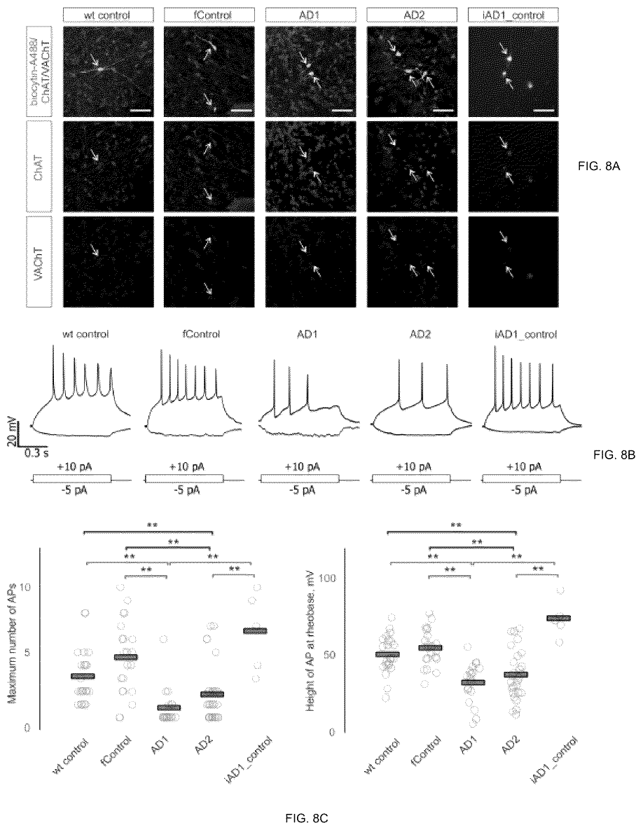

Using BFCN differentiation protocol, we were able to generate electrophysiologically active cholinergic neurons in a dish from two PSEN2^{N141I} mutant AD patients, wild-type and familial controls starting from differentiation day 35. We were initially unable to obtain mature action potential waveforms from BFCNs grown in neurobasal media at this stage, but switching to Brainphys' media significantly improved electrophysiological properties of cultured neurons [3]. These findings are in line with electrophysiological characterization of other iPSC generated neurons used to compare both media [3]. The benefits of the protocol containing Brainphysm media in two additional cell lines (including the H9 embryonic stem cell line) with comparable endpoint expression of ChAT and VAChT as well as electrophysiological responses were repeated. In order to investigate the electrophysiological properties of BFCN, we recorded from a total of 94 neurons (22 wild-type control, 21 familial control, 18 AD1, 28 AD2 and 5 iAD1_control) using whole cell patch-clamp method. In all experimental groups, recorded neurons displayed voltage-activated currents through sodium and potassium ion channels, ability to generate action potentials and displayed classical neuronal morphologies (FIGS. 7A-7B). In subset of experiments, recorded neurons were labeled with biocytin through a patch pipette, which allowed for post hoc cell identification and ICH characterization. We found that all biocytin-labelled cells were also immuno-positive for ChAT and VAChT (n=12, FIG. 8A).

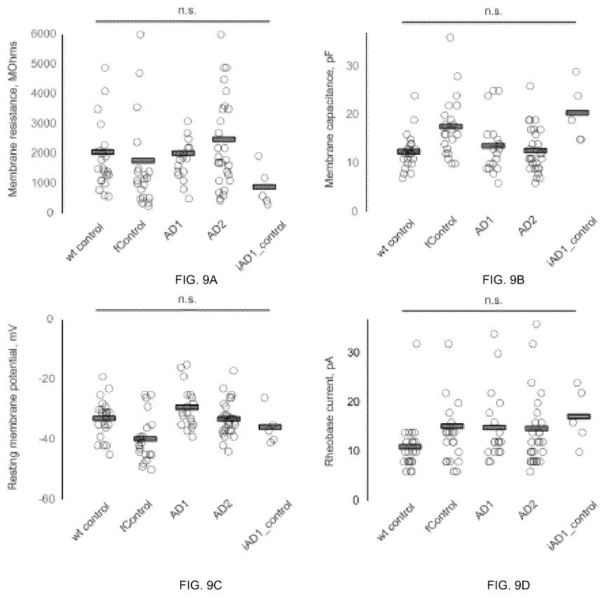

Significant differences between the groups in terms of neuronal membrane resistance and capacitance were not observed, membrane resting potential and the minimum current required for generation of a single action potential (FIGS. 9A-9D). However, it was observed that there were significant mutation-related, editing-reversible differences in excitability of BFCNs.

Neurons derived from AD1 and AD2 lines (as compared to WT and familial controls) were able to generate fewer maximum number of spikes in response to a square depolarizing current injection (ANOVA test with Tukey's post hoc comparisons, FIGS. 8B, 8C). Height of the first action potential at rheobase current injection was also significantly decreased in AD1 and AD2 BFCNs (FIG. 1C). Importantly, CRISPR/Cas9 correction of the PSEN2 point mutation in the AD1 mutant iPSC line abolished the observed electro-physiological deficit, restoring both the maximal number of spikes and spike height to the levels recorded in wild-type and familial controls (ANOVA test with Tukey's post hoc comparisons, FIGS. 8A-8C).

### Discussion

There are 5 million people currently affected by Alzheimer's disease in the US and, according to the Alzheimer's Association, this number will increase to 16 million by the year 2050. Unfortunately, we only have direct evidence for genetic causation that accounts for 3-5% of these patients. This percentage encompasses the EOFAD variants, caused by inherited fully penetrant autosomal dominant mutations in the amyloid protein precursor (APP), or PSEN1, PSEN2 that constitute the γ-secretase apparatus [87], and changes in their function increases the production of Aβ42 oligomers and/or deposition of amyloid plaques.

After decades studying murine models of AD that do not fully recapitulate the pathophysiology of this disease in the human brain [5, 57, 58], a complementary new concept of AD modeling in vitro has emerged upon the breakthrough by [81] allowing adult human tissue reprogramming into iPSC using defined factors, and their subsequent in vitro differentiation into specific brain cell types.

BFCNs are one of the most vulnerable neuronal populations whose deterioration explains, in part, the cognitive decline in AD patients. Apart from the evidence for BFCN failure and atrophy, other studies have revealed that human embryonic stem cell-derived BFCNs transplanted into AD mouse models can be associated with improvement in the learning behavior of the implanted mouse [94]. These findings highlight the relevance of iPSC- and ESC-derived BFCNs as not only early clinical indicators but also as a potential strategy for subtype-specific cell-based therapy for AD [39]. In order to move this cell-based therapeutic strategy forward, there has been an urgent need for a refined differentiation protocol to generate human ESC- and/or iPSC-derived BFCNs.

Our first goal was to develop an improved protocol for the generation of BFCNs and intermediate neural progenitors (NPCs), followed by the use of these methods when differentiating cell lines from both control subjects and those harboring the PSEN2^{N141I} mutation. Using fibroblasts isolated from 3 sisters, 2 carrying the PSEN2 mutation and displaying cognitive decline, with the third wild-type for the mutation, iPSCs were developed [60]. In order to approach the dissection of the fidelity of linkage of various phenotypes to the pathogenic mutation, we began by optimizing published BFCNs protocols [4, 17, 46, 50, 89] including the purification of an intermediate CD271+(p75) forebrain progenitor population by Fluorescence Activated Cell Sorting (FACS) to generate 3D ventralized neural embryoid bodies (vNEBs), which can be later dissociated to look at neuronal populations in a monolayer.

After induction of BFCN differentiation in these cell lines, we have analyzed: (1) capacity to generate Tuj1⁺/BF1⁺/ChAT⁺ neurons in vitro; (2) expression of genes/proteins of interest related to neuronal differentiation or inflammation; (3) generation of soluble and oligomeric Aβ40 and 42; (4) electrophysiological (ePhys) properties; and (5) selective vulnerability of BFCNs to one or more innate or microenvironmental factors within or in close approximation to those cells.

Several studies in AD mouse models highlight electrophysiological defects associated to late stages of AD pathology. Synaptic function in the hippocampus was reduced in APP23 mouse models [70]. Similarly, cholinergic neurons from the prefrontal cortex of TgCRND8 mice are unable to sustain cholinergic excitation as compared to control mice [64]. Here we report deficient electrophysiological properties in PSEN2^{N141I} iPSC-derived BFCNs in vitro. Notably, correction of this point mutation re-established neuronal excitability to the level of the control iPSC-derived neurons.

We have optimized an in vitro BFCN differentiation protocol from human iPSC, focusing on generating a homogeneous population of electrophysiologically active ChAT+/VAChT+ neurons in a reproducible and fast way. The innovations introduced to the protocol granted a homogeneous expression of Nkx2.1, a transcriptional marker for MGE subregions, as soon as day 8 and very robust by day 11, compared to day 20 suggested in previously published protocols [38]; in defined serum-free media conditions and without forcing overexpression of factors implicated in cholinergic fate. We were able to record mature action potentials in neurons from day 38 in culture, accompanying co-expression of cholinergic specific markers, which is an earlier time point as compared to other existing protocols using ES or iPSC [4, 17, 46, 50, 89]. Therefore, our protocol has potential application to high-throughput drug screening in homogeneous cholinergic cultures. In addition, the 3D structure of NEBs themselves if left undisssociated organoid form would also allow mechanistic analysis in a more physiological setting.

After applying this optimized protocol to PSEN2^{N141I} mutant iPSC lines, we found an increase in Aβ42/40 ratio in the conditioned media. We did not observe any evident defects in the neuronal differentiation process and expression of BFCN markers. Interestingly, we observed a decrease on BDNF gene expression in PSEN2^{N141I} NPCs, similar to results described in a report [18] wherein BDNF changes were observed in homozygous and heterozygous APP^{swe/}PSEN1^{M146V} mice. The two mutant lines are also carriers of one APOE E4 allele. The presence of this allelic variant, the most common and well characterized risk factor polymorphism for LOAD [16], may modulate the age of onset and severity of the phenotype [49]. Therefore, these iPSC lines combining both the EOFAD PSEN2 Volga mutation (or CRISPR/Cas9 corrected) and APOE E4 allele constitute a tremendously useful tool to study the pathophysiology of early onset AD in vitro, especially when apoE-secreting iPSC-derived astrocytes are also present.

Searching for adjacent mechanisms or events that may be a cause or a consequence of elevated β-amyloid production, researchers have found overactivated inflammation and electrophysiological defects associated with AD mutations. The concept of these defects being independent from β-amyloid deposition and their demonstration using CRISPR/Cas9 technology to correct EOFAD mutations would open the debate to the need of combined AD treatments not only targeting β-amyloid plaques (Gandy et al., in press), but also to overcome parallel inflammatory processes or excitotoxicity/defective neuronal firing.

NLRPs are components of the inflammasome, which induces the secretion of mature pro-inflammatory cytokine IL-13 in response to pathogens and toxic stimuli [11, 41]. NLRP2 appears dysregulated in astrocytes [45, 51], and NLRP3 in microglia [34] in the context of Alzheimer's disease as well as in other neurological diseases like Parkinson's disease [14, 32]; additionally, NLRP2/3 are altered in pathologies that show comorbidity with AD: obesity, type-2 diabetes. We previously reported an unexpected association of elevated expression of the inflammasome gene NLRP2 in iPSC-derived neurons from banked fibroblasts from subjects harboring PSEN1^{A246E} and PSEN1^{M146L} mutations [77]. This association reminded us of the association of the inflammatory skin disease acne inversa (AI) with mutations in presenilin 1, nicastrin, APH-1 and PEN-2, raising in our minds the question of whether some γ-secretase component mutations might be associated not only with proamyloidogenic actions but also with proinflammatory mechanisms.

Despite our observations PSEN2^{N141I} mutant cells had elevated NLRP2 compared to controls, we were not able to attribute this upregulation to the familial PSEN2 mutation, as gene correction did not significantly reduce NLRP2 levels. Our results suggest that, although inflammasome dysregulation may occur in the brains of EOFAD patients, there may be factors triggering this event apart from any effect of PSENs on inflammasome biology that are reflected in reprogrammed PSEN2 mutant cell lines. Some potential explanations for this PSEN2-independent NLRP2 upregulation include effects of the apoE4 allele present in both PSEN2 subjects (not preset in controls) or epigenetic effects on fibroblasts collected from the EOFAD subjects that are maintained through the reprogramming process.

Electrophysiological defects in neurons have been associated with PSEN1 and PSEN2 mutations. Some of these defects are attributed to altered function of voltage-gated K+ channels, potentially through the cleavage of channel components mediated by the PS/γ-secretase apparatus [44, 72]. Presenilin mutations also disrupt calcium signaling by increasing the levels of calcium stored in the endoplasmic reticulum that result in increased stimulus-induced released into the cytosol, rather than altered influx of calcium. One of the mechanisms behind neuronal calcium dysregulation was described in cortical neurons from PSEN1^{M146V} mice, mediated by inositol triphosphate (IP3) [79]; and, more directly, the formation of dual function protein-ion channels by unprocessed PSEN1 and PSEN2 themselves, modulating the exit of calcium from the endoplasmic reticulum [29, 55, 80, 84]. Given the important role of presenilins on potassium and calcium flux and neuronal excitability, mutations in PSEN1 and PSEN2 may lead to reduced neuronal excitability and neurotoxicity. Mice carrying mutant forms of APP exhibited aberrant action potentials associated to a decrease in sodium currents with no alteration in potassium currents, only after plaque burden was considerable [9]. There is evidence that APP overexpression causes hyperexcitability in mouse cortical neurons [75, 86, 92].

Mucke and Selkoe [52] have highlighted a toxic effect of A3 resulting in synaptic and network dysfunction. In fibroblasts and neural cell lines, A3-mediated accumulation of mitochondrial Ca²⁺ was elevated when mutant forms of PS1 were expressed [31]. Neuronal firing patterns in mouse hippocampal neurons were altered by exposure to A3 [67, 69]. A3 exposure was also associated with altered K⁺ channel conductance in pyramidal neurons [54]. PSEN1 mutations have been observed to associate with altered Ca²⁺ mitochondrial channels in the cerebellum, apparently causing reduced spike activity in Purkinje cells in the absence of amyloid plaque deposition [74]. Aβ42 may accentuate the defects present in Ca²⁺ homeostasis by modulation of additional voltage-dependent ion channels [8, 25, 76, 88].

Apart from mouse data and immortalized neuronal cell lines, electrophysiological defects in iPSC-derived neurons upon exposure to A3 have been shown: hiPSC-derived cortical pyramidal neurons and GABAergic interneurons have deficient action potentials upon exposure to A3 [56], and neurons differentiated from hiPSC harboring PS1^{A426E} mutation also showed deficient firing patterns [47]. However, there are no previously published data on characterization of electrophysiological properties of PSEN2 mutant iPSC-derived BFCNs.

Hyper- or hypoexcitatory effects and differences in firing frequency vary with the gene mutation and are highly dependent on the neuronal subtype [37, 48]. All these events may contribute to the progressive neurodegeneration present in the pathogenesis of AD, and we specifically document events that may account for the neuronal defects associated to early stages of EOFAD human pathogenesis. Here we report defective electrophysiological properties in iPSC-derived BFCNs that are specifically associated with the PSEN2N141I familial mutation. Interestingly, although some of the previous studies attribute this impairment in neuronal activity to the build-up of plaques in the brain of AD mice, we found a substantial impairment in the induced action potentials in the absence of amyloid plaques, solely in the presence of a discrete excess of Aβ42 oligomers in the culture media, consistent with other reports [18]. Correction of this point mutation re-established the firing patterns to those of the wildtype iPSC-derived neurons.

Modulators of potassium channels in neurons have proven efficacy in memory improvement in AD mouse models [44]. Modulation of Ca²⁺ channels and excitotoxicity may open a new wave of AD drugs. Understanding the mechanism through which PSEN2 mutations affect the electrophysiological activity in different subsets of neuronal populations and unraveling the connection between PSEN2, other genetic modulatory factors and inflammation will potentially lead to, not only alternative symptomatic treatments, but also to novel drugs decreasing the Ca²⁺-mediated vulnerability to ROS and potentially stopping the neuronal loss and progression of the disease, if administered at early stages.

It is clear that mutant presenilins alter neuronal excitability even before the formation of A3 plaques [18, 74]. One plausible hypothesis is that APP and presenilins may exert effects that modulate neuronal excitability through currently unrecognized mechanisms acting separate from their roles in the biogenesis of A3. Accumulation of A3 could synergize with the altered electrophysiological mechanisms in a pathway leading to AD. With the wealth of data supporting neuronal excitotoxicity as a key mechanism implicated in AD, further studies focusing on clarifying the possible role(s) of PSENs and/or A3 in physiological or pathological events are warranted.

### Conclusions

We have optimized an in vitro protocol to generate human BFCNs from iPSCs from presenilin 2 (PSEN2) mutation carriers and controls. As expected, PSEN2^{N141I} was associated with an increase in the Aβ42/40 in iPSC-derived BFCNs, and this was reversed by CRISPR/Cas9-mediated gene editing. Unexpectedly, iPSC-derived BFCNs or cortical neurons from PSEN2^{N141I} carriers showed diminished basal excitability as quantified by a reduction of both spike frequency and spike amplitude. This electrophysiological phenotype was also abolished following CRISPR/Cas9 correction of the PSEN2^{N141I} mutation. The gene editing data confirm that there was a robust consistency of mutation-related changes that characterized all the expected findings and genotypes from all cells.

### Abbreviations

AD: Alzheimer's Disease;
ApoE: Apolipoprotein E;
APP: Amyloid Protein Precursor;
AVG: Average;
A3: Amyloid Beta;
BDNF: Brain Derived Neurotrophic Factor;
BF1: Brain Factor 1;
BFCNs: Basal Forebrain Cholinergic Neurons;
ChAT: Acetylcholine Transferase;
DAPT: (N—[N-(3,5-difluorophenacetyl)-L-alanyl]-S-phenylglycine t-butyl ester);
DIV: Days in vitro;
DNA: Deoxyribonucleic Acid;
DPBS: Dulbecco's Phosphate-Buffered Saline;
DPBST: Dulbecco's Phosphate-Buffered Saline+0.1% Triton X-100;
EGTA: Ethylene-bis(oxyethylenenitrilo)tetraacetic acid;
EOFAD: Early Onset Familial Alzheimer's Disease;
ESC: Embryonic Stem Cells;
FACS: Fluorescence-Activated Cell Sorting;
GAPDH: Glyceraldehyde-3-Phosphate Dehydrogenase;
GFP: Green Fluorescent Protein;
HDR: Homology Directed Repair;
HFIP: 1,1,1,3,3,3-hexafluoro-2-propanol;
HRP: Horseradish Peroxidase;
IPSCs: Induced Pluripotent Stem Cells;
LDH: Lactate Dehydrogenase;
MAP2: Microtubule-Associated Protein 2;
MGE: Medial Ganglionic Eminences;
NEBs: Neuronal Embryoid Bodies;
NGF: Nerve Growth Factor;
NLRP2: NLR Family Pyrin Domain Containing 2;
NPC: Neural Progenitor Cells;
PFA: Paraformaldehyde;
PSEN: Presenilin;
RNA: Ribonucleic Acid;
Rock: Rho-Associated, coiled-coil containing protein kinase;
RT: Reverse Transcriptase;
RT-qPCR: Real-time quantitative polymerase chain reaction;
SAG: Smoothened Agonist;
SDS-PAGE: Sodium Dodecyl Sulfate PolyAcrylamide Gel Electrophoresis;
SEM: Standard Error of the Mean;
sgRNA: Single Guide RNA;
Shh: Sonic HedgeHog;
SNP: Single Nucleotide Polymorphism;
ssODN: Single Stranded Oligonucleotides;
TBST: Tris-Buffered Saline+0.1% Tween;
VACht: Vesicular Acetylcholine Transporter;
WT: Wild Type

### REFERENCES (EACH ARE INCORPORATED HEREIN BY REFERENCE)

1. Arendt T, Bigl V, Arendt A, Tennstedt A (1983) Loss of neurons in the nucleus basalis of Meynert in Alzheimer's disease, paralysis agitans and Korsakoff's disease. Acta Neuropathol 61:101-108
2. Armijo E, Gonzalez C, Shahnawaz M, Flores A, Davis B, Soto C (2017) Increased susceptibility to A3 toxicity in neuronal cultures derived from familial Alzheimer's disease (PSEN1^{A246E}) induced pluripotent stem cells. Neurosci Lett 639:74-81. doi:10.1016/j.neulet.2016.12.060
3. Bardy C, van den Hurk M, Eames T, Marchand C, Hernandez R V, Kellogg M, Gorris M, Galet B, Palomares V, Brown J, Bang A G, Mertens J, Böhnke L, Boyer L, Simon S, Gage F H (2015) Neuronal medium that supports basic synaptic functions and activity of human neurons in vitro. Proc Natl Acad Sci USA 112:E2725-E2734. doi:10.1073/pnas.1504393112
4. Bissonnette C J, Lyass L, Bhattacharyya B J, Belmadani A, Miller R J, Kessler J A (2011) The controlled generation of functional basal forebrain cholinergic neurons from human embryonic stem cells. Stem Cells 29:802-811. doi:10.1002/stem.626
5. Borchelt D R, Ratovitski T, van Lare J, Lee M K, Gonzales V, Jenkins N A, Copeland N G, Price D L, Sisodia S S (1997) Accelerated amyloid deposition in the brains of transgenic mice coexpressing mutant presenilin 1 and amyloid precursor proteins. Neuron 19:939-945. doi:10.1016/S0896-6273(00)80974-5
6. Bowen D M, Smith C B, White P, Davison A N (1976) Neurotransmitter-related enzymes and indices of hypoxia in senile dementia and other abiotrophies. Brain 99:459-496
7. Bragina O, Sergeieva S, Serg M, 2arkovsky T, Maloverjan A, Kogerman P, 2arkovsky A (2010) Smoothened agonist augments proliferation and survival of neural cells. Neurosci Lett 482:81-85. doi:10.1016/j.neulet.2010.06.068
8. Briggs C A, Schneider C, Richardson J C, Stutzmann G E (2013) Beta amyloid peptide plaques fail to alter evoked neuronal calcium signals in APP/PS1 Alzheimer's disease mice. Neurobiol Aging 34:1632-1643. doi:10.1016/j.neurobiolaging.2012.12.013
9. Brown J T, Chin J, Leiser S C, Pangalos M N, Randall A D (2011) Altered intrinsic neuronal excitability and reduced Na+ currents in a mouse model of Alzheimer's disease. Neurobiol Aging 32:2109.e1-2109.e14. doi:10.1016/j.neurobiolaging.2011.05.025
10. Brueggen K, Dyrba M, Barkhof F, Hausner L, Filippi M, Nestor P J, Hauenstein K, Klöppel S, Grothe M J, Kasper E, Teipel S J (2015) Basal forebrain and hippocampus as predictors of conversion to Alzheimer's disease in patients with mild cognitive impairment—a multicenter DTI and Volumetry study. J Alzheimers Dis 48:197-204. doi:10.3233/JAD-150063
11. Bruey J M, Bruey-Sedano N, Newman R, Chandler S, Stehlik C, Reed J C (2004) PAN1/NALP2/PYPAF2, an inducible inflammatory mediator that regulates NF-kappaB and caspase-1 activation in macrophages. J Biol Chem 279:51897-51907. doi:10.1074/jbc.M406741200
12. Chambers S M, Fasano C A, Papapetrou E P, Tomishima M, Sadelain M, Studer L (2009) Highly efficient neural conversion of human E S and iPS cells by dual inhibition of SMAD signaling. Nat Biotechnol 27:275-280. doi: 10.1038/nbt.1529
13. Chu V T, Weber T, Wefers B, Wurst W, Sander S, Rajewsky K, Kühn R (2015) Increasing the efficiency of homology-directed repair for CRISPR-Cas9-induced precise gene editing in mammalian cells. Nat Biotechnol 33:543-548. doi:10.1038/nbt.3198
14. Codolo G, Plotegher N, Pozzobon T, Brucale M, Tessari I, Bubacco L, de Bernard M (2013) Triggering of inflammasome by aggregated α-synuclein, an inflammatory response in synucleinopathies. PLoS One 8:e55375. doi:10.1371/journal.pone.0055375
15. Cong L, Ran F A, Cox D, Lin S, Barretto R, Habib N, Hsu P D, Wu X, Jiang W, Marraffini L A, Zhang F (2013) Multiplex genome engineering using CRISPR/Cas systems. Science 339:819-823. doi:10.1126/science.1231143
16. Corder E H, Saunders A M, Strittmatter W J, Schmechel D E, Gaskell P C, Small G W, Roses A D, Haines J L, Pericak-Vance M A (1993) Gene dose of apolipoprotein E type 4 allele and the risk of Alzheimer's disease in late onset families. Science 261:921-923
17. Crompton L A, Byrne M L, Taylor H, Kerrigan T L, Bru-Mercier G, Badger J L, Barbuti P A, Jo J, Tyler S J, Allen S J, Kunath T, Cho K, Caldwell M A (2013) Stepwise, non-adherent differentiation of human pluripotent stem cells to generate basal forebrain cholinergic neurons via hedgehog signaling. Stem Cell Res 11:1206-1221. doi:10.1016/j.scr.2013.08.002
18. Cummings D M, Liu W, Portelius E, Bayram S, Yasvoina M, Ho S-H, Smits H, Ali S S, Steinberg R, Pegasiou C-M, James O T, Matarin M, Richardson J C, Zetterberg H, Blennow K, Hardy J A, Salih D A, Edwards F A (2015) First effects of rising amyloid-3 in transgenic mouse brain: synaptic transmission and gene expression. Brain 138:1992-2004. doi:10.1093/brain/awv127
19. Danjo T, Eiraku M, Muguruma K, Watanabe K, Kawada M, Yanagawa Y, Rubenstein J L R, Sasai Y (2011) Subregional specification of embryonic stem cell-derived ventral Telencephalic tissues by timed and combinatory treatment with extrinsic signals. J Neurosci 31:1919-1933. doi:10.1523/JNEUROSCI.5128-10.2011
20. Davies P, Maloney A J (1976) Selective loss of central cholinergic neurons in Alzheimer's disease. Lancet (London, England). 2:1403
21. Davis K L, Mohs R C, Davis B M, Horvath T B, Greenwald B S, Rosen W G, Levy M I, Johns C A (1983) Oral physostigmine in Alzheimer's disease. Psychopharmacol Bull 19:451-453
22. De Strooper B After Solanezumab: Where Should Alzheimer's Research Go?ALZFORUM. 2017
23. Echeverria V, Berman D E, Arancio O (2007) Oligomers of beta-amyloid peptide inhibit BDNF-induced arc expression in cultured cortical neurons. Curr Alzheimer Res 4:518-521
24. Epelbaum S, Genthon R, Cavedo E, Habert M O, Lamari F, Gagliardi G, Lista S, Teichmann M, Bakardjian H, Hampel H, Dubois B (2017) Preclinical Alzheimer's disease: a systematic review of the cohorts underlying the concept. Alzheimers Dement. doi:10.1016/j.jalz.2016.12.003
25. Etcheberrigaray R, Ito E, Kim C S, Alkon D L (1994) Soluble beta-amyloid induction of Alzheimer's phenotype for human fibroblast K+ channels. Science 264:276-279
26. Flandin P, Zhao Y, Vogt D, Jeong J, Long J, Potter G, Westphal H, Rubenstein J L R (2011) Lhx6 and Lhx8 coordinately induce neuronal expression of Shh that controls the generation of interneuron progenitors. Neuron 70:939-950. doi:10.1016/j.neuron.201 1.04.020
27. Francis P T, Palmer A M, Snape M, Wilcock G K (1999) The cholinergic hypothesis of Alzheimer's disease: a review of progress. J Neurol Neurosurg Psychiatry 66:137-147. doi:10.1136/JNNP.66.2.137
28. Gamzu E R, Thal L J, Davis K L (1990) Therapeutic trials using tacrine and other cholinesterase inhibitors. Adv Neurol 51:241-245
29. Gandy S, Doeven M K, Poolman B (2006) Alzheimer disease: presenilin springs a leak. Nat Med 12:1121-1123. doi:10.1038/nm1006-1121
30. Goulbum A L, Alden D, Davis R P, Micallef S J, Ng E S, Yu Q C, Lim S M, Soh C-L, Elliott D A, Hatzistavrou T, Bourke J, Watmuff B, Lang R J, Haynes J M, Pouton C W, Giudice A, Trounson A O, Anderson S A, Stanley E G, Elefanty A G (2011a) A targeted NKX2.1 human embryonic stem cell reporter line enables identification of human basal forebrain derivatives. Stem Cells 29:462-473. doi:10.1002/stem.587
31. Guo Q, Fu W, Holtsberg F W, Steiner S M, Mattson M P (1999) Superoxide mediates the cell-death-enhancing action of presenilin-1 mutations. J Neurosci Res 56:457-470. doi:10.1002/(SICI)1097-4547(19990601)56:5<457::AID-JNR2>3.0.CO; 2-P
32. Gustot A, Gallea J I, Sarroukh R, Celej M S, Ruysschaert J-M, Raussens V (2015) Amyloid fibrils are the molecular trigger of inflammation in Parkinson's disease. Biochem J 471:323-333. doi:10.1042/BJ20150617
33. Hager K, Baseman A S, Nye J S, Brashear H R, Han J, Sano M, Davis B, Richards H M (2014) Effects of galantamine in a 2-year, randomized, placebo-controlled study in Alzheimer's disease. Neuropsychiatr Dis Treat 10:391-401. doi:10.2147/NDT.557909
34. Halle A, Hornung V, Petzold G C, Stewart C R, Monks B G, Reinheckel T, Fitzgerald K A, Latz E, Moore K J, Golenbock D T (2008) The NALP3 inflammasome is involved in the innate immune response to amyloid-. Nat Immunol 9:857-865. doi:10.1038/ni.1636
35. Heine V M, Griveau A, Chapin C, Ballard P L, Chen J K, Rowitch D H (2011) A small-molecule smoothened agonist prevents Glucocorticoid-induced neonatal Cerebellar injury. Sci Transl Med 3:105ra104. doi:10.1126/scitranslmed.3002731
36. Hixson J E, Vernier D T (1990) Restriction isotyping of human apolipoprotein E by gene amplification and cleavage with HhaI. J Lipid Res 31:545-548. doi:10.0000/PMID2341813
37. Hoxha E, Boda E, Montarolo F, Parolisi R, Tempia F (2012) Excitability and synaptic alterations in the cerebellum of APP/PS1 mice. PLoS One 7:e34726. doi:10.1371/journal.pone.0034726
38. Hu Y, Qu Z, Cao S, Li Q, Ma L, Krencik R, Xu M, Liu Y (2016) Directed differentiation of basal forebrain cholinergic neurons from human pluripotent stem cells. J Neurosci Methods 266:42-49. doi:10.1016/j.jneumeth.2016.03.017
39. Hunsberger J G, Rao M, Kurtzberg J, Bulte J W M, Atala A, LaFerla F M, Greely H T, Sawa A, Gandy S, Schneider L S, Doraiswamy P M (2016) Accelerating stem cell trials for Alzheimer's disease. Lancet Neurol 15:219-230. doi:10.1016/S1474-4422(15)00332-4
40. Jack C R, Wiste H J, Weigand S D, Knopman D S, Lowe V, Vemuri P, Mielke M M, Jones D T, Senjem M L, Gunter J L, Gregg B E, Pankratz V S, Petersen R C (2013) Amyloid-first and neurodegeneration-first profiles characterize incident amyloid PET positivity. Neurology 81:1732-1740. doi:10.1212/01.wnl.0000435556.21319.e4
41. Kinoshita T, Wang Y, Hasegawa M, Imamura R, Suda T (2005) PYPAF3, a PYRIN-containing APAF-1-like protein, is a feedback regulator of caspase-1-dependent interleukin-1beta secretion. J Biol Chem 280:21720-21725. doi:10.1074/jbc.M410057200
42. Kruglikov I, Rudy B (2008) Perisomatic GABA release and thalamocortical integration onto neocortical excitatory cells are regulated by neuromodulators. Neuron 58:911-924. doi:10.1016/j.neuron.2008.04.024
43. Levy-Lahad E, Wasco W, Poorkaj P, Romano D, Oshima J, Pettingell W, Yu C, Jondro P, Schmidt S, Wang K et al (1995) Candidate gene for the chromosome 1 familial Alzheimer's disease locus. Science 269:973-977. doi:10.1126/science.7638622
44. Liu D, Pitta M, Lee J-H, Ray B, Lahiri D K, Furukawa K, Mughal M, Jiang H, Villarreal J, Cutler R G, Greig N H, Mattson M P (2010) The KATP channel activator diazoxide ameliorates amyloid- and tau pathologies and improves memory in the 3×TgAD mouse model of Alzheimer's disease. J Alzheimers Dis 22:443-457. doi:10.3233/JAD-2010-101017
45. Liu L, Chan C (2014) The role of inflammasome in Alzheimer's disease. Ageing Res Rev 15:6-15. doi:10.1016/j.arr.2013.12.007
46. Liu Y, Weick J P, Liu H, Krencik R, Zhang X, Ma L, Zhou G, Ayala M, Zhang S-C (2013) Medial ganglionic eminence-like cells derived from human embryonic stem cells correct learning and memory deficits. Nat Biotechnol 31:440-447. doi:10.1038/nbt.256
47. Mahairaki V, Ryu J, Peters A, Chang Q, Li T, Park T S, Burridge P W, Talbot C C, Asnaghi L, Martin L J, Zambidis E T, Koliatsos V E, Koliatsos V E (2014) Induced pluripotent stem cells from familial Alzheimer's disease patients differentiate into mature neurons with amyloidogenic properties. Stem Cells Dev 23:2996-3010. doi:10.1089/scd.2013.0511
48. Marcantoni A, Raymond E F, Carbone E, Marie H (2014) Firing properties of entorhinal cortex neurons and early alterations in an Alzheimer's disease transgenic model. Pflugers Arch 466:1437-1450. doi:10.1007/s00424-013-1368-z
49. Marchani E E, Bird T D, Steinbart E J, Rosenthal E, Yu C-E, Schellenberg G D, Wijsman E M (2010) Evidence for three loci modifying age-at-onset of Alzheimer's disease in early-onset PSEN2 families. Am J Med Genet B Neuropsychiatr Genet 153B:1031-1041. doi:10.1002/ajmg.b.31072
50. Maroof A M, Keros S, Tyson J A, Ying S-W, Ganat Y M, Merkle F T, Liu B, Goulburn A, Stanley E G, Elefanty A G, Widmer H R, Eggan K, Goldstein P A, Anderson S A, Studer L (2013) Directed differentiation and functional maturation of cortical interneurons from human embryonic stem cells. Cell Stem Cell 12:559-572. doi:10.1016/j.stem.2013.04.008
51. Minkiewicz J, de Rivero Vaccari J P, Keane R W (2013) Human astrocytes express a novel NLRP2 inflammasome. Glia 61:1113-1121. doi:10.1002/glia.22499
52. Mucke L, Selkoe D J (2012) Neurotoxicity of amyloid-protein: synaptic and network dysfunction. Cold Spring Harb Perspect Med 2:a006338. doi:10.1101/cshperspect.a006338
53. Müller U, Winter P, Graeber M B (2011) Alois Alzheimer's case, Auguste D., did not carry the N141I mutation in &lt; emph type=&quot; ital&quot; &gt; PSEN2&lt; /emph&gt; characteristic of Alzheimer disease in Volga Germans. Arch Neurol 68:1210. doi:10.1001/archneurol.2011.218
54. Nava-Mesa M O, Jimenez-Diaz L, Yajeya J, Navarro-Lopez J D (2013) Amyloid-induces synaptic dysfunction through G protein-gated inwardly rectifying potassium channels in the fimbria-CA3 hippocampal synapse. Front Cell Neurosci 7:117. doi:10.3389/fncel.2013.00117
55. Nelson O, Supnet C, Tolia A, Horre K, De Strooper B, Bezprozvanny I (2011) Mutagenesis mapping of the Presenilin 1 calcium leak conductance pore. J Biol Chem 286:22339-22347. doi:10.1074/jbc.M111.243063
56. Nieweg K, Andreyeva A, van Stegen B, Tanriöver G, Gottmann K (2015) Alzheimer's disease-related amyloid-induces synaptotoxicity in human iPS cell-derived neurons. Cell Death Dis 6:el 709. doi:10.1038/cddis.2015.72
57. Oddo S, Caccamo A, Shepherd J D, Murphy M P, Golde T E, Kayed R, Metherate R, Mattson M P, Akbari Y, LaFerla F M (2003) Triple-transgenic model of Alzheimer's disease with plaques and tangles: intracellular Abeta and synaptic dysfunction. Neuron 39:409-421
58. Onos K D, Sukoff Rizzo S J, Howell G R, Sasner M (2016) Toward more predictive genetic mouse models of Alzheimer's disease. Brain Res Bull 122:1-11. doi:10.1016/j.brainresbull.2015.12.003
59. Oyama F, Sawamura N, Kobayashi K, Morishima-Kawashima M, Kuramochi T, Ito M, Tomita T, Maruyama K, Saido T C, Iwatsubo T, Capell A, Walter J, Grünberg J, Ueyama Y, Haass C, Ihara Y (1998) Mutant presenilin 2 transgenic mouse: effect on an age-dependent increase of amyloid beta-protein 42 in the brain. J Neurochem 71:313-322
60. Paull D, Sevilla A, Zhou H, Hahn A K, Kim H, Napolitano C, Tsankov A, Shang L, Krumholz K, Jagadeesan P, Woodard C M, Sun B, Vilboux T, Zimmer M, Forero E, Moroziewicz D N, Martinez H, Malicdan M C V, Weiss K A, Vensand L B, Dusenberry C R, Polus H, Sy K T L, Kahler D J, Gahl W A, Solomon S L, Chang S, Meissner A, Eggan K, Noggle S A (2015) Automated, high-throughput derivation, characterization and differentiation of induced pluripotent stem cells. Nat Methods 12:885-892. doi: 10.1038/nmeth.3507
61. Perry E K, Gibson P H, Blessed G, Perry R H, Tomlinson B E (1977) Neurotransmitter enzyme abnormalities in senile dementia. Choline acetyltransferase and glutamic acid decarboxylase activities in necropsy brain tissue. J Neurol Sci 34:247-265
62. Pini L, Pievani M, Bocchetta M, Altomare D, Bosco P, Cavedo E, Galluzzi S, Marizzoni M, Frisoni G B (2016) Brain atrophy in Alzheimer's disease and aging. Ageing Res Rev 30:25-48. doi:10.1016/j.arr.2016.01.002
63. Price D L, Tanzi R E, Borchelt D R, Sisodia S S (1998) Alzheimer's disease: genetic studies and transgenic models. Annu Rev Genet 32:461-493. doi:10.1146/annurev.genet.32.1.461
64. Proulx É, Fraser P, McLaurin J, Lambe E K (2015) Impaired cholinergic excitation of prefrontal attention circuitry in the TgCRND8 model of Alzheimer's disease. J Neurosci 35:12779-12791. doi:10.1523/JNEUROSCI.4501-14.2015
65. Pruszak J, Sonntag K C, Aung M H, Sanchez-Pernaute R, Isacson O (2007) Markers and methods for cell sorting of human embryonic stem cell-derived neural cell populations. Stem Cells 25(9):2257-2268 Epub 2007 Jun. 21. PubMed PMID: 17588935; PubMed Central PMCID: PMC2238728
66. Ran F A, Hsu P D, Wright J, Agarwala V, Scott D A, Zhang F (2013) Genome engineering using the CRISPR-Cas9 system. Nat Protoc 8:2281-2308. doi:10.1038/nprot.2013.143
67. Ren S-C, Shao H, Ji W-G, Jiang H-H, Xu F, Chen P—Z, Mi Z, Wen B, Zhu G-X, Zhu Z-R (2015) Riluzole prevents soluble a₁₋₄₂ oligomers-induced perturbation of spontaneous discharge in the hippocampal CA1 region of rats. Amyloid 22:36-44. doi:10.3109/13506129.2014.990558
68. Richardson C D, Ray G J, DeWitt M A, Curie G L, Com J E (2016) Enhancing homology-directed genome editing by catalytically active and inactive CRISPR-Cas9 using asymmetric donor DNA. Nat Biotechnol 34:339-344. doi:10.1038/nbt.3481
69. Ripoli C, Cocco S, Li Puma D D, Piacentini R, Mastrodonato A, Scala F, Puzzo D, D'Ascenzo M, Grassi C (2014) Intracellular accumulation of Amyloid-(a) protein plays a major role in a-induced alterations of Glutamatergic synaptic transmission and plasticity. J Neurosci 34:12893-12903. doi:10.1523/JNEUROSCI.1201-14.2014
70. Roder S, Danober L, Pozza M, Lingenhoehl K, Wiederhold K—H, Olpe H-R (2003) Electrophysiological studies on the hippocampus and prefrontal cortex assessing the effects of amyloidosis in amyloid precursor protein 23 transgenic mice. Neuroscience 120:705-720. doi:10.1016/50306-4522(03)00381-6
71. Rylett R J, Ball M J, Colhoun E H (1983) Evidence for high affinity choline transport in synaptosomes prepared from hippocampus and neocortex of patients with Alzheimer's disease. Brain Res 289:169-175
72. Sachse C C, Kim Y H, Agsten M, Huth T, Alzheimer C, Kovacs D M, Kim D Y (2013) BACE1 and presenilin/-secretase regulate proteolytic processing of KCNE1 and 2, auxiliary subunits of voltage-gated potassium channels. FASEB J27:2458-2467. doi:10.1096/fj.12-214056
73. Sawamura N, Morishima-Kawashima M, Waki H, Kobayashi K, Kuramochi T, Frosch M P, Ding K, Ito M, Kim T W, Tanzi R E, Oyama F, Tabira T, Ando S, Ihara Y (2000) Mutant-presenilin 2-transgenic mice: a large increase in the levels of a beta 42 is presumably associated with the low-density membrane domain that contains decreased levels of glycerophospholipids and sphingomyelin. J Biol Chem 275:27901-27908. doi: 10.1074/jbc.M004308200
74. Sepulveda-Falla D, Barrera-Ocampo A, Hagel C, Korwitz A, Vinueza-Veloz M F, Zhou K, Schonewille M, Zhou H, Velazquez-Perez L, Rodriguez-Labrada R, Villegas A, Ferrer I, Lopera F, Langer T, De Zeeuw C I, Glatzel M (2014) Familial Alzheimer's disease-associated presenilin-1 alters cerebellar activity and calcium homeostasis. J Clin Invest 124:1552-1567. doi:10.1172/JC166407
75. Šišková Z, Justus D, Kaneko H, Friedrichs D, Henneberg N, Beutel T, Pitsch J, Schoch S, Becker A, von der Kammer H, Remy S (2014) Dendritic structural degeneration is functionally linked to cellular Hyperexcitability in a mouse model of Alzheimer's disease. Neuron 84:1023-1033. doi:10.1016/j.neuron.2014.10.024
76. Smilansky A, Dangoor L, Nakdimon I, Ben-Hail D, Mizrachi D, Shoshan-Barmatz V (2015) The voltage-dependent Anion Channel 1 mediates Amyloidtoxicity and represents a potential target for Alzheimer disease therapy. J Biol Chem 290:30670-30683. doi:10.1074/jbc.M115.69149
77. Sproul A A, Jacob S, Pre D, Kim S H, Nestor M W, Navarro-Sobrino M, Santa-Maria I, Zimmer M, Aubry S, Steele J W, Kahler D J, Dranovsky A, Arancio O, Crary J F, Gandy S, Noggle S A (2014) Characterization and molecular profiling of PSEN1 familial Alzheimer's disease iPSC-derived neural progenitors. PLoS One 9:e84547. doi:10.1371/journal.pone.0084547
78. Stine W B, Dahlgren K N, Krafft G A, LaDu M J (2003) In vitro characterization of conditions for amyloid-beta peptide oligomerization and fibrillogenesis. J Biol Chem 278:11612-11622. doi:10.1074/jbc.M210207200
79. Stutzmann G E, Caccamo A, LaFerla F M, Parker I (2004) Dysregulated IP3 signaling in cortical neurons of knock-in mice expressing an Alzheimer's-linked mutation in Presenilin1 results in exaggerated Ca2+ signals and altered membrane excitability. J Neurosci 24:508-513. doi:10.1523/JNEUROSCI.4386-03.2004
80. Supnet C, Bezprozvanny I (2011) Presenilins function in E R calcium leak and Alzheimer's disease pathogenesis. Cell Calcium 50:303-309. doi:10.1016/j.ceca.2011.05.013
81. Takahashi K, Yamanaka S (2006) Induction of Pluripotent stem cells from mouse embryonic and adult fibroblast cultures by defined factors. Cell 126(4):663-676. doi:10.1016/j.cell.2006.07.024
82. Thal L J, Fuld P A, Masur D M, Sharpless N S (1983) Oral physostigmine and lecithin improve memory in Alzheimer disease. Ann Neurol 13:491-496. doi:10.1002/ana.410130504
83. Tomita T, Maruyama K, Saido T C, Kume H, Shinozaki K, Tokuhiro S, Capell A, Walter J, Grunberg J, Haass C, Iwatsubo T, Obata K (1997) The presenilin 2 mutation (N141I) linked to familial Alzheimer disease (Volga German families) increases the secretion of amyloid beta protein ending at the 42nd (or 43rd) residue. Proc Natl Acad Sci USA 94:2025-2030
84. Tu H, Nelson O, Bezprozvanny A, Wang Z, Lee S—F, Hao Y—H, Semeels L, De Strooper B, Yu G, Bezprozvanny I (2006) Presenilins form ER Ca2+ leak channels, a function disrupted by familial Alzheimer's disease-linked mutations. Cell 126:981-993. doi:10.1016/j.cell.2006.06.059
85. Tucker E S, Segall S, Gopalakrishna D, Wu Y, Vernon M, Polleux F, Lamantia A-S (2008) Molecular specification and patterning of progenitor cells in the lateral and medial ganglionic eminences. J Neurosci 28:9504-9518. doi:10.1523/JNEUROSCI.2341-08.2008
86. Varga E, Juhász G, Bozsó Z, Penke B, Fülöp L, Szegedi V (2014) Abeta(1-42) enhances neuronal excitability in the CA1 via NR2B subunit-containing NMDA receptors. Neural Plast 2014:1-12. doi:10.1155/2014/584314
87. Verdile G, Gandy S E, Martins R N (2007) The role of presenilin and its interacting proteins in the biogenesis of Alzheimer's beta amyloid. Neurochem Res 32:609-623. doi:10.1007/s11064-006-9131-x
88. Wang X, Zhang X-G, Zhou T-T, Li N, Jang C—Y, Xiao Z—C, Ma Q-H, Li S (2016) Elevated neuronal excitability due to modulation of the voltage-gated Sodium Channel Nav1.6 by A 1-42. Front Neurosci 10:94. doi:10.3389/fnins.2016.00094
89. Wicklund L, Leão R N, Stromberg A-M, Mousavi M, Hovatta O, Nordberg A, Marutle A (2010) β-amyloid 1-42 oligomers impair function of human embryonic stem cell-derived forebrain cholinergic neurons. PLoS One 5:e15600. doi:10.1371/journal.pone.0015600
90. Wolfe M S, Selkoe D J, Xia W, Ostaszewski B L, Diehl T S, Kimberly W T (1999) Two transmembrane aspartates in presenilin-1 required for presenilin endoproteolysis and [gamma]-secretase activity. Nature 398:513-517. doi:10.1038/19077
91. Xu H, Sakiyama-Elbert S E (2015) Directed differentiation of V3 Interneurons from mouse embryonic stem cells. Stem Cells Dev 24:2723-2732. doi:10.1089/scd.2015.0122
92. Xu W, Fitzgerald S, Nixon R A, Levy E, Wilson D A (2015) Early hyperactivity in lateral entorhinal cortex is associated with elevated levels of A PP metabolites in the Tg2576 mouse model of Alzheimer's disease. Exp Neurol 264:82-91. doi:10.1016/j.expneurol.2014.12.008
93. Yagi T, Ito D, Okada Y, Akamatsu W, Nihei Y, Yoshizaki T, Yamanaka S, Okano H, Suzuki N (2011) Modeling familial Alzheimer's disease with induced pluripotent stem cells. Hum Mol Genet 20:4530-4539. doi:10.1093/hmg/ddr394
94. Yue W, Li Y, Zhang T, Jiang M, Qian Y, Zhang M, Sheng N, Feng S, Tang K, Yu X, Shu Y, Yue C, Jing N (2015) ESC-derived basal forebrain cholinergic neurons ameliorate the cognitive symptoms associated with Alzheimer's disease in mouse models. Stem Cell Rep 5:776-790. doi:10.1016/j.stemcr.2015.09.01.

Although the invention has been described with reference to the examples herein, it will be understood that modifications and variations are encompassed within the spirit and scope of the invention. Accordingly, the invention is limited only by the following claims.

What is claimed is:

1. A method of generating basal forebrain cholinergic neurons (BFCNs) comprising: culturing pluripotent stem cells (PSCs) in a basal media comprising an inhibitor of transforming growth factor beta (TGF-β) signaling and an activator of sonic hedgehog (Shh) signaling to induce neuroectodermal differentiation, wherein the basal media lacks basic fibroblast growth factor (bFGF), TGF-β, lithium chloride (Li—Cl), GABA and pipecolic acid, thereby generating BFCNs.

2. A method of treating a disease or disorder in a subject comprising administering a BFCN generated using the method of claim 1 to the subject, thereby treating the disease or disorder.

3. The method of claim 2, wherein the disease or disorder is an amyloidogenic disease.

4. The method of claim 2, wherein the disease or disorder is associated with diminished neuronal excitability in BFCNs of the subject.

5. The method of claim 3, wherein the disease or disorder is selected from the group consisting of systemic amyloidosis, Alzheimer's disease, mature onset diabetes, Parkinson's disease, Huntington's disease, fronto-temporal dementia, and prion-related transmissible spongiform encephalopathies.

6. The method of claim 2, wherein the BFCN comprises a genome repaired PSEN2 mutation.

7. The method of claim 6, wherein the PSEN2 mutation is PSEN2^{N141I}.

8. A method of restoring neuronal excitability in basal forebrain cholinergic neurons (BFCNs) in a subject, comprising:
a) isolating a BFCN from the subject, wherein the BFCN has a mutation in presenilin 2 (PSEN2) resulting in impaired neuronal excitability of the BFCN;
b) generating an induced pluripotent stem cell (iPSC) using the BFCN of (a);
c) repairing the PSEN2 mutation in the iPSC;
d) culturing the iPSC of (c) using the method of claim 1 to generate a BFCN having the repaired mutation; and
e) administering the iPSC of (d) to the subject, thereby restoring neuronal excitability in BFCNs in the subject.

9. The method of claim 8, wherein the mutation is PSEN2^{N141I}.

10. The method of claim 8, wherein the subject has, or is at risk of having Alzheimer's disease.

11. A method of identifying a compound for treatment or prevention of a disease or disorder associated with diminished neuronal excitability in basal forebrain cholinergic neurons (BFCNs), the method comprising:
a) contacting a BFCN or neuronal embryoid body (NEB) generated by the method of claim 1 with a candidate compound, wherein the BFCN comprises a mutation in presenilin 2 (PSEN2) resulting in impaired neuronal excitability of the BFCN; and
b) detecting neuronal excitability of the BFCN after contact with the candidate compound.

12. The method of claim 11, wherein the mutation is PSEN2^{N141I}.

13. The method of claim 11, wherein the disease or disorder is Alzheimer's disease.

14. A basal forebrain cholinergic neuron (BFCN) generated using the method of claim 1.

15. The BFCN of claim 14, wherein the genome has a recombinantly introduced marker.

16. A kit for generating a basal forebrain cholinergic neuron (BFCN) comprising a culture media having an inhibitor of transforming growth factor beta (TGF-β) signaling and an activator of sonic hedgehog (Shh) signaling, wherein the basal media lacks basic fibroblast growth factor (bFGF), TGF-β, lithium chloride (Li—Cl), GABA and pipecolic acid, wherein the inhibitor comprises SB431542 and LDN193189, and wherein the activator comprises Smoothened Agonist (SAG) and purmorphamine.

17. The kit of claim 16, wherein the kit further comprises reagents for generating an induced pluripotent stem cell (iPSC).

18. The kit of claim 16, wherein the kit further comprises a gene-editing reagent.

19. The kit of claim 16, wherein the kit further comprises a reagent for detecting a genetic mutation.

20. The kit of claim 19, wherein the mutation is PSEN2^{N141I}.
